(19) United States
(12) Patent Application Publication
(10) Pub. No.: US 2023/0172984 A1
(43) Pub. Date: Jun. 8, 2023

(54) CLEARANCE OF SENESCENT CELLS BY ACTIVATION OF iNKT CELLS

(71) Applicant: THE REGENTS OF THE UNIVERSITY OF CALIFORNIA, Oakland, CA (US)

(72) Inventor: Anil Bhushan, San Francisco, CA (US)

(21) Appl. No.: 17/919,677

(22) PCT Filed: Apr. 22, 2021
(86) PCT No.: PCT/US2021/028724

§ 371 Date: Oct. 18, 2022

Related U.S. Application Data
(60) Provisional application No. 63/014,694, filed on Apr. 23, 2020.

(51) Int. Cl. A61K 35/17; A61K 31/7028; A61P 3/10

(52) U.S. Cl. CPC A61K 35/17; A61K 31/7028; A61P 3/10

(57) ABSTRACT

Activated iNKT cells act as senolytic agents, removing senescent cells from target tissues, organs, and compartments of the body. Provided are methods of clearing pathological accumulations of senescent cells by administration of iNKT cell activators such as an alpha-galactosylceramide, or variants or by the adoptive transfer of iNKT cells or precursors, enabling the treatment of senescence-associated conditions such as diabetes, lung fibrosis, and other conditions.

### CROSS-REFERENCE TO RELATED APPLICATIONS

This application claims the benefit of priority to U.S. Provisional Patent Application Ser. No. 63/014,694 entitled “Clearance of Senescent Cells by Activation of iNKT Cells,” filed Apr. 23, 2020, the contents of which are hereby incorporated by reference.

### STATEMENT REGARDING FEDERALLY SPONSORED RESEARCH OR DEVELOPMENT

Not Applicable.

### BACKGROUND OF THE INVENTION

Cellular senescence is a process wherein the cell cycle is arrested and cells enter a functional but non-dividing state. It is believed that this this cellular pathway evolved to protect against genetic instability and cancer in cells that have accumulated DNA-damaging insults, and thus senescence prevents unrestricted cell proliferation. There is also evidence that senescence acts against viral infections, inhibiting the replicative processes that are hijacked by viruses.

Senescent cells produce numerous cytokines and chemokines. Over short time scales, this response is thought to be adaptive, wherein the proinflammatory signals recruit immune elements to the afflicted area, enabling immune surveillance of potentially oncogenic or infected cells. In healthy and young individuals, it is believed that the senescent cells are efficiently removed by the immune system.

However, in diseased or aged individuals, it appears that immune system removal of senescent cells is impaired. Under such conditions, senescent cells accumulate and the proinflammatory action of these cells can become pathologic. Senescent cells accumulate in ever greater numbers over time, and the signaling molecules they secrete become deleterious to surrounding tissues, causing inflammation, remodeling tissue in aberrant ways, and potentially fostering the onset of cancer rather than suppressing it. The presence of senescent cells likely worsens the progression of many age-related diseases and processes. In some cases, an extended state of unresolved stress causes the senescent cells to exhibit what is known as the SASP, for senescence-associated secretory phenotype. SASP cells secrete a number of inflammatory signal molecules, including cytokines, chemokines, and other factors. This SASP secretome may profoundly affect neighboring, non-senescent cells, impairing their function and facilitating their transition to senescent cells as well.

Accumulation of senescent cells is thus believed to underlie a great number of disease conditions and age-related pathologies. For example, accumulation of senescent cells has been implicated in formation of lesions such as atherosclerotic plaques, in diabetes, in neurodegeneration, and other conditions. Accordingly, the use of senolytics has been proposed for the prevention and treatment of conditions mediated by senescent cells.

Senolytics are compositions that selectively remove senescent cells, by various mechanisms. Exemplary senolytics include BCl inhibitors such as navitoclax and venetoclax, which have been tested against multiple forms of cancer, such as certain leukemias, with positive results. However, these drugs have various side effects, including testicular toxicity and thrombocytopenia. Accordingly, there remains a need in the art for new classes of senolytic agents that can safely facilitate the removal of senescent cells.

Meanwhile, invariant natural killer T cells (iNKTs) are a specialized subset of T cells found in circulation and also as resident cells in various tissues, organs, or other compartments of the body of an organism. The iNKT T-cell receptor (TCR) is formed by highly restricted somatic recombination, resulting in an invariant TCR repertoire. iNKT cells are implicated in various immune system functions, such as responding to microbial infection and anti-tumor immunity, and have also been implicated in immune system dysfunction, as in asthma, allergies, and autoimmune conditions.

Various treatment methods are in development for the modulation of iNKT cell numbers and activity. In the context of treating cancer, activated iNKT cells rapidly secrete both Th1 and Th2 cytokines and activate NK and other immune cells to stimulate anti-tumor immune responses. Accordingly, numerous tumor immunity treatments acting by activation of iNKT cells are under investigation and development.

The use of iNKT activation in other contexts has also been explored. In one study, iNKT activation was used to treat pulmonary fibrosis in a mouse model, wherein it was concluded that IFN-γ-producing NKT cells had anti-fibrotic activity in pulmonary fibrosis by regulating TGF-β1 production, as described in Kim, 2005. Natural killer T (NKT) cells attenuate bleomycin-induced pulmonary fibrosis by producing interferon-gamma. The American journal of pathology, 167(5), 1231-1241. In the context of liver fibrosis, it was found that natural killer cells preferentially kill senescent activated stellate cells in vitro and in vivo, thereby facilitating the resolution of fibrosis, as described in Krizhanovsky et al., 2008. Senescence of activated stellate cells limits liver fibrosis. Cell, 134(4), 657-667. In the context of treating microbial infections, the activation of iNKT cells has been found to augment innate and acquired immune processes, for example, as described in Kinjo et al., 2018. Functions of CD1d-Restricted Invariant Natural Killer T Cells in Antimicrobial Immunity and Potential Applications for Infection Control, Front Immunol. 9: 1266. Activation of iNKT cells with alpha-GalCer has been found to reverse adverse metabolic phenotypes in the HFD mouse model, for example, as described in Lynch et al., 2012. Adipose Tissue Invariant NKT Cells Protect against Diet-Induced Obesity and Metabolic Disorder through Regulatory Cytokine Production. Immunity 37, 574-587; Schipper et al., 2012. Natural killer T cells in adipose tissue prevent insulin resistance. J. Clin. Invest. 122, 3343-3354; Ji et al., 2012. Activation of natural killer T cells promotes M2 macrophage polarization in adipose tissue and improves systemic glucose tolerance via interleukin-4 (IL-4)/STAT6 protein signaling axis in obesity. J. Biol. Chem. 287, 13561-13571. While therapeutic iNKT activation has been explored in these various contexts, no connection between iNKT activation and senescent cells has been previously reported.

### SUMMARY OF THE INVENTION

The inventors of the present disclosure have advantageously discovered means of promoting immune-system clearance of senescent cells. The inventors of the present disclosure have made the unprecedented discovery that activation of certain iNKTs achieves the removal of pathological accumulations of senescent cells.

In a first aspect, the scope of the invention encompasses novel methods of clearing senescent cells by the activation of iNKT cells. Activation of iNKT cells may be achieve by the use of various iNKT-activating ligands, including glycolipids. In an alternative method, the abundance of activated iNKT cells in a target tissue, organ, or compartment increased by the administration of iNKT cells or iNKT cell precursors.

By the clearance of senescent cells, various preventative and therapeutic effects are achieved, including a reduction in the abundance of senescent cells, relief from the SASP phenotype, and the prevention and treatment of disease and age-related conditions mediated by the accumulation of senescent cells.

In one aspect, the scope of the invention encompasses methods of treating conditions associated with senescent cells by the therapeutic administration of iNKT cell activating agents. In the many embodiments disclosed herein, the treated condition may encompass various inflammatory or autoimmune conditions, neurodegenerative conditions, cardiovascular conditions, and age-related conditions.

The various implementations of the invention are described in detail next.

### BRIEF DESCRIPTION OF THE DRAWINGS

FIGS. 1A, 1B, 1C, and 1D. Senescent preadipocytes are defined by high SA-βgal activity and accumulate in white adipose tissue of HFD mice. FIG. 1A: qRT-PCR of senescence markers on CD45-depleted eWAT SVF cells from chow and HFD mice. Data are mean±SEM from n=6 mice for each group. FIG. 1B: CD1d expression on the eWAT SVF isolated from chow and HFD mice. The cells were stained with antibodies for CD45 and CD31 (to gate out immune and endothelial cells) along with the fluorogenic substrate C₁₂FDG and anti-CD1d antibody. The senescent preadipocytes (DAPI-CD45-CD31⁻ C12FDG^{Hi}) were then gated for relative CD1d expression. Quantification of CD1d expression from the CD45⁻ CD31⁻ C12FDG^{Hi} subset from chow and HFD mice is shown in the right panel. Data are represented as mean±SD from n=4 per experiment. FIG. 1C: Quantification of C₁₂FDG⁺ cells from chow or HFD mice. Data are mean±SD from n=4 mice per group. FIG. 1D: Quantification of the C₁₂FDG MFI of the entire CD45⁻ CD31⁻ population in the same chow and HFD mice as in (g). Data are mean±SD from n=4 mice per group. *p<0.05, **p<0.005, ***p<0.0005, two-tailed T-tests.

FIGS. 2A, 2B, 2C, and 2D. Senescent preadipocytes with high SA-βgal activity express SASP genes and are preferentially eliminated with senolytic treatment. FIG. 2A: C₁₂FDG MFI of the C₁₂FDG^{Lo} and C₁₂FDG^{Hi} populations. Data are mean±SD from n=3 mice per group. FIG. 2B: qRT-PCR of senescence markers in C₁₂FDG^{Hi} relative to C₁₂FDG^{Lo} cells from the same HFD mice. Data are mean±SD from n=3 mice per group. FIG. 2C: Quantification of the positive cells in the C₁₂FDG^{Hi} subset from vehicle and ABT-737 treated HFD mice. FIG. 2D: Quantification of the positive cells in the C₁₂FDG^{Lo} subset from vehicle and ABT-737 treated HFD mice. Data are mean±SD from n=3 mice per group. *p<0.05, **p<0.005, ***p<0.0005, two-tailed T-tests.

FIGS. 3A, 3B, 3C, and 3D. Activation of iNKT cells leads to clearance of senescent cells in white adipose tissue of HFD mice. FIG. 3A: Representative histograms of C₁₂FDG fluorescence and quantification of the C₁₂FDG^{Hi} subpopulation of CD45⁻ cells from Chow (n=4), HFD Ctrl (n=5) and HFD+GC (n=5) mice per group. Data are mean±SD. FIG. 3B: Glucose tolerance test (GTT) was performed at 10 days after α-GalCer injection. Mice were administered 2 g/kg glucose via i.p. injection after a 14 hr fast. Blood glucose was measured at 0, 15, 30, 60, and 120 after glucose injection. Data are mean±SEM from n=8-9 mice per group. FIG. 3C: HOMA-IR was assessed at 10 days after α-GalCer injection based on the fasting blood glucose and fasting insulin concentration using the equation, HOMA-IR=(glucose in mmol/L×insulin in mIU/mL)/22.5. Data are mean±SEM from n=8-9 mice per group. FIG. 3D: Quantification of the percent of C₁₂FDG^{Hi} subpopulation from the CD45⁻ CD31⁻ subset for each group of mice. Data are mean±SD from n=2 experiments. *p<0.05, **p<0.005, ***p<0.0005, one-way ANOVA.

FIGS. 4A, 4B, 4C, 4D, 4E, 4F, and 4G. Activation of iNKT cells preferentially eliminates senescent cells in a murine lung injury model and in vitro using human cells. FIG. 4A: Representative histogram of C₁₂FDG fluorescence and quantifications of mean±SD from n=3 mice per group in CD45⁻ lung parenchymal cells. FIG. 4B: qRT-PCR of senescence and SASP in CD45-depleted lung cells, showing mean±SD of n=3 mice per group, *p<0.05, one-way ANOVA. FIG. 4C: Percentages of iNKT cells as a proportion of T cells from the same mice as (b), mean±SD of n=2 mice per group. FIG. 4D: Quantification of fibrosis by hydroxyproline (HP) concentrations in Control (n=5), Bleo (n=10), and Bleo+GC (n=10) mice per group, error bars are SD. ***p<0.0005, *p<0.05, one-way ANOVA Krusak-Wallis test. FIG. 4E: Kaplan Meier survival curve for mice injured with intratracheal bleomycin (4 U/kg) and injected with α-GalCer or vehicle at day 5 (arrow). N=10 mice per group. Significance calculated by Log-rank (Mantel-Cox) test. FIG. 4F: Time course of cytolysis assay showing mean percent of cytolysis over 18 hours of the assay for the indicated samples. Each time-point measurement was from n=3 biological replicates for each sample. FIG. 4G: Mean percent cytolysis at 8 h and 18 h for indicated 1:2 target-effector ratios (n=3 biological replicates each), error bars are SD. *p<0.05, ***p<0.0005 one-way ANOVA.

### DETAILED DESCRIPTION OF THE INVENTION

The various inventions disclosed herein are based on the unprecedented discovery that activated iNKTs achieve clearance of senescent cells, which results in the prevention and treatment of pathological conditions associated with the accumulation of senescent cells. The clearance may be achieved by the direct action of iNKT cells and/or by the action of other cells recruited by iNKT surveillance.

iNKT Cells. iNKT cells have invariant αβ TCRs that recognize glycolipid antigens presented by CD1d. CD1d is a non-classical MHC protein expressed by antigen-presenting cells, such as hematopoietic cells such as dendritic cells, B cells, T cells, and macrophages and is also expressed by various endothelial and epithelial cells. Self and foreign glycolipids in such cells are presented at the cell surface by CD1d. If the presented ligand is an activating species, it will bind to the invariant TCR of the iNKT cell and will activate various cytotoxic and/or immune responses.

In humans, iNKT cells express the invariant Vα24-Jα18 chains. In mice, iNKT cells expresses the Vα14-Jα18 TCRα chains. iNKT cells may be characterized functionally by their reactivity to alpha-Galactosylceramide (GalCer, as discussed below). Among the iNKT cell population, various subtypes are found, including iNKT1, iNKT2, iNKT17, iNKT10, and memory follicular helper iNKT (iNKT_{FH}) cells. The iNKT types are distinguished from one another by TCR affinity and activation response. Without being bound to a particular theory of operation, it is hypothesized by the inventor of the present disclosure that the clearance of senescent cells is largely achieved by iNKT1 cells. iNKT1 cells are a subset of iNTK cells that may be characterized by their production, upon activation, of a Th-1 type response, for example, the production of large amounts of IFNγ and Il-2 and little production of IL-4. In a primary embodiment, the iNKT cells by which the methods disclosed herein are iNKT1 cells. However, it will be understood that in alternative implementations, other types of iNKT cells, including iNKT2, iNKT17, iNKT10, and memory follicular helper iNKT (iNKT_{FH}) cells, may be activated or employed.

I. iNKT AGENTS

The various inventions disclosed herein are directed to what will be referred to herein as iNKT agents. As used herein, “iNKT agent” means an agent which promotes the clearance of senescent cells in a target organ, tissue, or compartment of the body by the direct and/or indirect action of iNKT cells. iNKT action may be direct, wherein cytotoxic activity by activated iNKT cells kills senescent cells. INKT action may be indirect, wherein activated iNKT cells recruit other immune elements that kill senescent cells, or induce apoptosis in senescent cells. An iNKT agent may achieve increased clearance of senescent cells by any number of mechanisms, including by activating resident iNKT cells, increasing the number of iNKT cells, and/or recruiting iNKT cells to the target.

In a primary implementation, the iNKT agent is an iNKT activator. An iNKT activator is a composition of matter that activates iNKT cells, for example, iNKT1 cells.

Activation of iNKT cells results in the rapid release of immune factors, and the activated cells proliferate at a high rate. INKT activators may include any number of compositions, primarily glycolipids such as GalCer and variants thereof. In other embodiments, the iNKT agent is a composition of matter that increases the recruitment of iNKT1 cells to the target organ, tissue, or compartment. In other embodiments, the iNKT agent is a cell, comprising an iNKT1 cell or iNKT1 cell precursor. The various iNKT agents utilized in the practice of the invention are described in more detail next.

iNKT Activators. In a primary embodiment, the iNKT agent of the invention is an iNKT activator. As defined above, iNKT activators are compositions of matter that act to activate iNKT cells, for example, iNKT1 cells. The activator will typically act by interaction with the TCR of the iNKT cell, wherein binding to the receptor initiates the robust extracellular secretion of immune factors by the cell and rapid cell division.

Activation of iNKT1 cells results in a Type-1 inflammatory response, including the secretion of interferon-gamma (IFNγ), interleukin (IL)-2, and tumor necrosis factor-α. In some embodiments, the activated iNKT1 cells induce a dominant Th1 response with little or no Th2 response. In some embodiments, the activated cells of the invention induce a mixed Th1 and Th2 response. The iNKT response is generally rapid, with cytokine secretion commencing within hours of activation, such that iNKT cells are considered a “first responder” to viral and other infections.

In various embodiments, iNKT activation may encompass any of: a measurable increase in immune factor secretion, for example, increased (IFNγ), interleukin (IL)-2 production; a measurable increase in a selected immune response, such as Th-1 responses; a measurable increase in iNKT cell abundance; and, a measurable increase iNKT cell proliferation rate. The increase may be any increase, for example, an increase of at least 5%, at least 10%, at least 20%, at least 30%, at least 40%, at least 50%, at least 60%, at least 70%, at least 80%, or at least 90%, for example, increase being an increase compared to the selected measure of iNKT activation in like, unactivated iNKT cells.

GalCer and Variants. A well-known iNKT activator is alpha-Galactosylceramide (GalCer), also known as KRN7000. Composition 1:

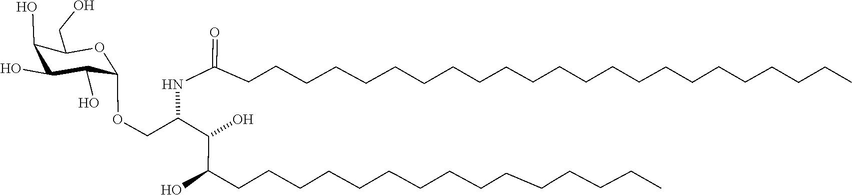

In various embodiments, the iNKT activator is a “GalCer variant,” the GalCer variant comprising a composition that shares structural similarity to GalCer and which also has iNKT1 activation properties. The structure of GalCer consists of an α-galactose bound by a glycosidic bond to a C18 phytosphingosine base with an amide-linked, saturated C26 fatty acyl chain. Galcer variants include modifications to the sphingosine chain, the N-acyl chain, the glycosidic bond and in the carbohydrate head moiety. Examples of GalCer analogs include: branched acyl chain α-GalCer analogs with improved solubility over GalCer; fluorinated 3′,4′-dideoxy α-GalCer analogs, such as 4′-deoxy α-GalCer analog; α-C-GalCer; α-S-GalCer; ThrCer; PBS-57; Nu-α-GalCer; and carbocyclic versions of α-GalCer such as RCAI-56.

In some embodiments, the activator may be a GalCer derivative in which galactose, a key element of iNKT TCR recognition, is substituted with a nonglycosidic substitutions, for example, as described in Silk et al., Cutting Edge: Nonglycosidic CD1d Lipid Ligands Activate Human and Murine Invariant NKT Cells, J Immunol 2008; 180:6452-6456. Exemplary GalCer variants include threitolceramide arabinitolceramide, and glycerolceramide. In one embodiment, the iNKT activator is threitolceramide modified to have restricted flexibility in the sugar headgroup, for example, by incorporating the threitol unit into a carbocycle such as six- or seven-membered ring, for example, as described in Jukes et al., Non-glycosidic compounds can stimulate both human and mouse iNKT cells, Eur. J. Immunol. 2016. 46: 1224-1234.

In one embodiment, the iNKT activator is a GalCer variant comprising GalCer having a modification at a fatty acyl chain or a terminal benzene ring at the phytosphingosine chain, for example, glycolipid, 7DW8-5, as described by Li et al., Design of a potent CD1d-binding NKT cell ligand as a vaccine adjuvant PNAS, 2010, 107: 3010-13015.

In one embodiment, the iNKT activator is a GalCer variant comprising carbohydrate and sphingoid base modifications in an alpha-galactosyl ceramide, for example, as described in Chennamadhavuni et al., Dual Modifications of α-Galactosylceramide Synergize to Promote Activation of Human Invariant Natural Killer T Cells and Stimulate Anti-tumor Immunity, 2018, Cell Chemical Biology 25, 571-584.

In one embodiment, the iNKT activator is a GalCer variant comprising an E-alkene linker between the sugar and lipid moieties, for example, GCK152, which has an aromatic ring in the tail of the acyl chain, for example, as described in Li et al., Invariant TCR Rather Than CD1d Shapes the Preferential Activities of C-Glycoside Analogues Against Human Versus Murine Invariant NKT Cells, J Immunol 2009; 183:4415-4421.

Other iNKT Activators. The iNKT activator may encompass other glycolipid activators of iNKT cells. In one embodiment, the iNKT activator is a synthetic aminocyclitolic ceramide, such as HS44, for example, as described in Kerzerho et al., Structural and functional characterization of a novel non-glycosidic iNKT agonist with immunomodulatory properties J Immunol. 2012 Mar. 1; 188(5): 2254-2265.

In one embodiment, the iNKT activator comprises glycosphingolipid, α-glyco(Gal/Glc)diacylglycerol, β-glucosylsphingosine, phosphatidylinositol, phos-phatidylethanolamine, phosphatidylglycerol, diphosphati-dylglycerol, lysophosphatidylcholine, or phenyl 2,2,4,6,7-pentamethyldihydrobenzofuran-5-sulfonate, for example, as described in Macho-Fernandez and Brigl, The extended family of CD1d-restricted NKT cells: sifting through a mixed bag of TCRs, antigens, and functions, Front. Immun. 2015, Vol. 6, Article 362.

In some embodiments, the iNKT activator is a naturally occurring glycolipid antigen derived from a microbial pathogen that acts as an activator of iNKT cells, for example: glycosylceramides from Sphingomonadaceae bacterial species, including for example, GSL-1, GSL-1′, GSL-3 and GSL-4; galactosyl diacylglycerols isolated from Borrelia burgdorferi; galactosyl diacylglycerols from Streptococcus pneumoniae; α-Linked glucosyl DAGs and a disaccharide Gal-Glu-DAG of the protozoan Entamoeba histolytica; asperamide B from Aspergillus spp.; α-mannnosyl1-3 (6′-O-acyl α-mannosyl)-1-1 monoacylglycerol from Saccharopolyspora; cholesteryl 6′-O-acyl α-mannoside, found Candida albicans; and PI57 from Helicobacter pylori.

In some embodiments, the iNKT activator is an endogenous mammalian glycolipid presented by CD1d, for example, isoglobotrihexosylceramide, β-glucosylceramide, lysophosphotidylcholine, and lysosphingomelin.

In some implementations, the iNKT activator is an antibody which binds to the TCR of iNKT cells and, by such binding, causes their activation. Exemplary antibodies include the 6B11 monoclinal antibody against the iNKT TCR-alpha CDR3 loop, disclosed in Exley et al, Selective activation, expansion, and monitoring of human iNKT cells with a monoclonal antibody specific for the TCR α-chain CDR3 loop, Eur. J. Immunol. 2008. 38: 1756-1766. Additional iNKT activating antibodies include NKTT320, a recombinant, humanized, monoclonal antibody that binds selectively and with high affinity to human iNKT TCR, as described in Patel et al., 2020, Cancer Immunotherapeutic Potential of NKTT320, a Novel, Invariant, Natural Killer T Cell-Activating, Humanized Monoclonal Antibody, Int J Mol Sci. 2020 June; 21: 4317.

In some embodiments, the iNKT activator is a small molecule activator of iNKT cells.

iNKT Cells as iNKT Agents. In some implementations, an increase in iNKT cells in the target organ, tissue, or compartment is achieved by the transfer of iNKT cells or iNKT cell precursors to the targeted organ, tissue, or compartment. In this implementation iNKT cells or precursors are derived from a source; expanded; and then introduced into the targeted tissue, organ, or compartment of a subject. The cells may be activated prior to transfer or following transfer by the use of one or more selected iNKT activators.

In some embodiments, the iNKT cells are autologous cells, isolated from a subject, expanded, and then introduced back into the same subject. In an alternative implementation, the iNKT cells are allogenic cells, derived from an individual other than the subject.

The use of transplanted iNKT cells for cancer immunotherapy is known. Accordingly, there are multiple methods known in the art for the isolation, expansion, and transplantation of iNKT cells. Clinical trials using adoptive cell transfer of expanded iNKT cells are reviewed, for example, in Wolf et al., Novel Approaches to Exploiting Invariant NKT Cells in Cancer Immunotherapy, Front. Immun. 2018, Volume 9, Article 384.

Isolation of iNKT cells from biological materials such as peripheral blood may be achieved by various means. iNKT cells may be isolated by FACS selection of cells exhibiting iNKT markers, for example, CD3, CD4, and CD8. Alternatively, glycolipid-loaded CD1d dimers or tetramers may be used as selection tools, for example, as described in Watarai et al., Methods for detection, isolation and culture of mouse and human invariant NKT cells, Nature Protocols, 2008, 3:70-77. Alternatively, antibodies selective for iNKT cells may be used to isolate them, for example, an anti-Vα24Jα18 CDR3 loop antibody, as described in Exley et al, Selective activation, expansion, and monitoring of human iNKT cells with a monoclonal antibody specific for the TCR α-chain CDR3 loop, Eur. J. Immunol. 2008. 38: 1756-1766.

Following isolation, iNKT cells may be efficiently expanded ex vivo by any number of platforms known in the art. Cells may be cultured in an appropriate medium such as RPMI 1640 medium with pulsed addition of an iNKT activator to spur high rates of proliferation. Exemplary methods of expanding iNKTs are described, for example, in Chiba et al., Rapid and reliable generation of invariant natural killer T-cell lines in vitro, Immunology, 128, 324-333.

In a variation of the adoptive transfer method, the administered cells may be iNKT precursors, being cells which differentiate into or produce iNKT cells subsequent in vivo subsequent to infusion. For example, the use of engineered hematopoietic stem cells that develop into iNKT cells has been demonstrated in the context of cancer treatment, for example, as described by Zhu et al., Development of Hematopoietic Stem Cell-Engineered Invariant Natural Killer T Cell Therapy for Cancer, Cell Stem Cell 25: 542-557.

iNKT Agent Pharmaceutical Compositions. The selected iNKT agent may be presented to target tissues, organs, or compartments in various forms and formulations. In some implementations, the selected iNKT activator is combined with additional compositions for improved delivery and activity.

The pharmaceutical compositions may be formulated for efficient delivery by a selected route. In various implementations, the iNKT activators are formulated for administration any number of routes, including, for example, oral, intravenous, intra-arterial, intraperitoneal, intrapulmonary, oral, inhalation, intravesicular, intramuscular, intra-tracheal, subcutaneous, intraocular, intrathecal, transmucosal, and transdermal delivery.

The pharmaceutical compositions of the invention will comprise any number of components suitable for, and which facilitate delivery by, the selected delivery route. The iNKT activators may be formulated in combination with pharmaceutically acceptable excipients, carriers, diluents, release formulations and other drug delivery or drug targeting vehicles, as known in the art. In one embodiment, the iNKT activator pharmaceutical composition comprises the selected therapeutic agent admixed with a polymeric material for timed release elution of the agent, for example, to prevent premature digestion of the material in the digestive tract. In one embodiment, the iNKT activator is coated onto an implant or drug-eluting device.

In some implementations, the selected iNKT activator is complexed with CD1d proteins for example, recombinantly produced CD1d oligomers such as CD1d dimers or tetramers. Exemplary methods of preparing such CD1d oligomers is found in Karadimitris et al., 2001, Human CD1d-glycolipid tetramers generated by in vitro oxidative refolding chromatography, PNAS Mar. 13, 2001 98 (6) 3294-3298.

In some implementations, the selected iNKT activator is loaded into a vesicle or like vesicular body, such as an exosome, to facilitate delivery, for example, as described in Gehrmann et al., 2013. Synergistic induction of adaptive antitumor immunity by codelivery of antigen with alpha-galactosylceramide on exosomes. Cancer Res. (2013) 73:3865-76.

In one embodiment, the iNKT activators are loaded onto or otherwise formulated with nanoparticles containing or functionalized with the selected active agent, for delivery by nanoparticle-based delivery methods. For example, poly(lactic-coglycolic acid) (PLGA)-based nanoparticles loaded with GalCer were demonstrated by Ferandez et al., 2014 Targeted Delivery of α-Galactosylceramide to CD8α⁺ Dendritic Cells Optimizes Type I NKT Cell-Based Antitumor Responses to promote strong anti-tumour iNKT activity, J Immunol 193: 961-969;

In various implementations, the pharmaceutical compositions of the invention may comprise a targeting moiety that preferentially directs the selected iNKT agent to the target tissue, organ, or compartment of the body. In some embodiments, the targeting moiety may comprise an antibody or antigen-binding fragment thereof which selectively binds epitopes found in the target tissue or cell type. For example, Fernandez et al, cited above, demonstrated targeted delivery of GalCer loaded nanoparticles to CD8+ cells by functionalization with an antibody chains against CD205. In some embodiments, the pharmaceutical composition comprises a CD1 protein or oligomer fused to an antigen-binding moiety such as an antibody or antibody-binding fragment thereof, wherein the antigen-binding moiety promotes delivery of the iNKT agent to a selected target cell type, for example, as demonstrated in Corgnac et al., 2013 CD1d-antibody fusion proteins target iNKT cells to the tumor and trigger long-term therapeutic responses. Cancer Immunol Immunother. (2013) 62:747-60.

In some implementations, the selected iNKT activator is provided on a cell that has been loaded with the selected agent. iNKT activating glycolipids may be loaded onto or presented by CD1d expressing cells, including dendritic cells, monocyte-derived dendritic cells or other monocyte-derived antigen-presenting cells. Exemplary methods of producing, loading, and delivering such iNKT activator-functionalized cells are described in Toura et al., 1999. Cutting edge: inhibition of experimental tumor metastasis by dendritic cells pulsed with alpha-galactosylceramide. J Immunol (1999) 163:2387-91; Chang et al., 2005. Sustained expansion of NKT cells and antigen-specific T cells after injection of alpha-galactosyl-ceramide loaded mature dendritic cells in cancer patients. J. Exp. Med. 201: 1503-1517; and Fujii et al., 2002. Prolonged IFN-gamma-producing NKT response induced with alpha-galactosylceramide-loaded DCs. Nat. Immunol. 3: 867-874.

II. Therapeutic Clearance of Senescent Cells by iNKT Agents.

The various inventions disclosed herein are directed to the clearance of senescent cells for the treatment and prevention of diseases and other conditions by increasing iNKT numbers and activity. In a first embodiment, the scope of the invention encompasses an iNKT agent for use in a method of reducing the number of senescent cells in a target organ, tissue or compartment of the body of a subject. In this method, an iNKT agent is administered to the subject in a therapeutically effective amount sufficient to increase iNKT activity in the target, resulting in a reduction in the number of senescent cells therein.

In a second embodiment, the scope of the invention encompasses an iNKT agent for use in a method of reducing the pathological effects of senescent cells in a target organ, tissue or compartment of the body of a subject. In this method, an iNKT agent is administered to the subject in a therapeutically effective amount sufficient to increase iNKT activity in the target, resulting in a reduction in the number of senescent cells therein and amelioration of the pathological effects of the senescent cells. In some embodiments, the pathological effects of senescent cells are SASP-associated effects and are ameliorated by the enhanced removal of SASP cells by iNKT cells.

In a third embodiment, the scope of the invention encompasses an iNKT agent for use in a method of treating a senescence-associated condition in a subject. In this method, an iNKT agent is administered to the subject to increase iNKT senescent-cell clearing activity in the target, resulting in a reduction in the number of senescent cells therein and providing a therapeutic effect against the senescence-associated condition.

In a primary embodiment, the scope of the invention encompasses a method of treating a senescence-associated condition in a subject in need of treatment therefore by the administration to the subject of a therapeutically effective amount of an iNKT agent. In a related embodiment, the scope of the invention encompasses an iNKT agent for use in a method of treating a senescence-associated condition. In another aspect, the scope of the invention encompasses a method of using an iNKT agent in the manufacture of a medicament for use in a method treating a senescence-associated condition. The various methods of the invention are described next.

Senescent Cells. The methods of the invention encompass administration of iNKT agents to reduce the numbers of senescent cells in a target tissue, organ, or compartment. Senescent cells encompass any number of pathologic characteristics associated with cellular senescence. Senescent cells include cells having any number of markers or characteristics, including cell-cycle arrest, the upregulation of negative modulators of the cell cycle, chromatin “scars” and Senescence-associated heterochromatin foci, lipid deposits, telomere shortening, inductions of g-H2AX nuclear foci, upregulated p15, p16ink4a, p21 p53, and ADP-ribosylation factor (ARF), senescence-associated beta galactosidase (SA-β-gal), lipofuscin Aggregates in liposomes, and other markers known in the art as indicators of senescent cell identity.

Certain senescent cells acquire the SASP phenotype. SASP cells are senescent cells that exhibit a paracrine pro-inflammatory or pro-senescent effects on neighboring, non-senescent cells. SASP factors include drivers of SASP such as bromodomain and extra-terminal motif (BET) proteins (e.g. BRD2, BRd3, and BRD4), p38MAPK, JAK, NF-κB, and CCAAT-enhancer-binding proteins (C/EBP). SASP factors may further include secreted SASP factors in the cell environment, for example, secreted factors such as IL-6, Serpine1, G-CSF, Ccl2, Mmp9, Mmp12, Igfpb3, IL1, IL8, CXCL1, CXCL2, monocyte chemotactic protein 3, insulin-like growth factor-binding proteins (including IGFBP2, IGFBP3, IGFBP4, IGFBP5, and PGFBP6), colony stimulating factor, MMP-3, MMP-10, and serine proteases. SASP cells can be defined as those expression or exhibiting any known biomarkers of the SASP condition.

Subjects. The methods of the invention are directed to the prevention and treatment of various conditions associated with the pathological accumulation of senescent cells in a subject. The subject may be a human subject, for example, in some contexts, a patient. The subject may further comprise a non-human animal of any species, including test animals, veterinary subjects, pets, and livestock, for example, any of mice, rats, dogs, cats, sheep, goats, cows, pigs, horses, non-human primates, or other animals.

In various embodiments, the subject is a subject that has a senescence-associated condition as described below, for example, a subject that is need of treatment for an enumerated condition. In various embodiments, the subject is a subject at risk of acquiring, progressing to, or otherwise having an enumerated condition, for example, a subject in need of preventative treatment.

The ability to clear senescent cells appears to decline in individual as they age. In certain embodiments, the subject is an aged subject, for example, subject of at least 40 years of age, at least 50 years of age, or at least 60 years of age. In the case of non-human animal subjects, aged subjects will be defined according to criteria appropriate for the species of the subject. For example, mice of 18-24 months of age may correlate with humans aged about 55-70 years.

Administration of iNKT Agents. The iNKT agents are administered to subjects in a therapeutically effective amount, being an amount sufficient to induce a measurable selected biological effect and/or selected therapeutic effect. In a primary embodiment, the therapeutically effective amount is an amount sufficient to measurably reduce the numbers of senescent cells in the target. For example, in various embodiments, the reduction may be a reduction of at least 5%, at least 10%, at least 15%, at least 20%, at least 25%, at least 30%, at least 35%, at least 40%, at least 50%, at least 60%, at least 70%, at least 80%, at least 90%, or may comprise a substantially complete elimination of senescent cells, compared to the target prior to treatment or compared to like, untreated targets. in other implementations, the therapeutically effective amount is an amount sufficient to achieve a measurable treatment outcome, as described below.

The precise dosage of the iNKT agent will be determined according to the pharmacological properties of the iNKT agent, delivery route, target site characteristics, therapeutic needs, and other factors taken into account. Exemplary dosages are in the range of 0.1 ng to 1 mg/kg, for example, 1-100 μg/kg. An alternative dosage range is 50-5,000 micrograms per square meter of body surface, for example, as described in Giaccone et al. A phase I study of the natural killer T-cell ligand alpha-galactosylceramide (KRN7000) in patients with solid tumors. Clin Cancer Res. 2002; 8:3702-3709. In the case of iNKT agents comprising cells, such as iNKT cells, iNKT cell precursors, or CD1d-expressing cells loaded with iNKT activators (e.g. Galcer), exemplary therapeutically effective dosages for humans may be in the range of 100-500 million cells per treatment.

The timing of administration will be determined according to condition to be treated, the properties of the selected agent, and the desired therapeutic outcome. In a general implementation, the methods disclosed herein are applied in order to clear or substantially remove deleterious senescent cells from the target. In some embodiments, a single treatment is applied to achieve this outcome. In other implementations, several treatments are applied to achieve a reduction of senescent cells. In some cases, iNKT agents are administered at regular intervals to keep senescent cell numbers below a pathological level, for example, administration being, weekly, or monthly, quarterly, biannually, or annually, depending on therapeutic factors. In some implementations, wherein activity of iNKT agent is to be maintained at a desired and steady level, administration may be multiple times per day or daily. However, in general, it is only necessary to clear accumulations of senescent cells periodically, for example, at four week or monthly intervals.

In limited implementations of the methods disclosed herein, systemic application of iNKT agents is performed. However, As has been found in the case of systemically administered senolytics, systemic ablation of senescent cells can have significant and deleterious off-target effects. Accordingly, in most implementations, the methods of the invention are directed to selectively or preferentially reducing the numbers of senescent cells in a target tissue, organ, or compartment of the body wherein the accumulation of senescent cells is causing deleterious effects. The target may comprise any of a In tissue, organ, cell type, compartment, or other division of the body. For example, in various embodiments, as used herein, the target may encompass peripheral blood, the lymph system, the brain, kidney, liver, pancreas, lung; adipose tissue; white adipose tissue; brown adipose tissue; skeletal muscle; heart; pancreas; kidney; intestine; colon; hypothalamus; cardiac tissue; liver; bladder, spleen; lymph node; dermis; stomach; lung; pancreas, brain; ocular tissue; auditory canal or cells the ear such as hair cells; spinal cord; heart, esophagus, sinus tissues; testis, ovary, bone, peripheral nerve, cartilage, soft tissue, an element of the circulatory system, hair follicles, epidermis, reproductive organs; digestive tract, bladder, airway, the hemopoietic compartment or bone marrow; the entire subject, or any other selected tissue, organ, or cell type tissue, organ, or compartment may be any present in the body of the subject.

Localized administration to the target may be performed. In other implementations, the agent is delivered to the circulatory system and is transported to the target. In some implementations, or targeting moieties achieve preferential or selective delivery to the target. In other implementations, prodrug formulations promote selective activation of the iNKT agent in the target. The iNKT agents may be administered by any number of routes, including, for example, oral, intravenous, intra-arterial, intraperitoneal, intrapulmonary, oral, inhalation, intravesicular, intramuscular, intra-tracheal, subcutaneous, intraocular, intrathecal, intradermal, transmucosal, and transdermal delivery. Exemplary administration techniques include subcutaneous injection into adipose tissues, inhalation delivery by a dry powder inhaler or nebulizer, percutaneous delivery with catheters, and other methods known in the art for the delivery of agents to a target tissue, organ, or compartment of the body.

Treatment and Therapeutic Effect. The scope of the invention encompasses various methods of treating what will be referred to herein as a “senescence associated-condition.” A senescence-associated condition encompasses any state or condition associated with the accumulation of senescent cells. The scope of the invention is directed to the treatment of such senescence-associated conditions. As used herein, “treatment” means achieving any preventative or therapeutic effect.

In some embodiments, the therapeutic effect is an increase in the abundance of iNKT cells. An increase in the abundance of iNKT cells may encompass the increase of iNKT cells in a selected target comprising a tissue, organ, compartment, or other division of the body. In related embodiments, the therapeutic effect is an increase in the activation of iNKT cells, for example, measured as an increase in the number or proportion of iNKT cells that are activated, or by the activity level of iNKT cells in the selected compartment.

In some embodiments, the therapeutic effect is a reduction in the abundance of senescent cells in the subject, for example, wherein a therapeutic effect comprises a reduction in the number of senescent cells in a selected compartment. The reduction may be an absolute reduction in cell numbers, or a proportional reduction in the ratio of senescent cells to non-senescent cells. In various embodiments, a therapeutic effect is defined as a reduction in senescent cells in the selected compartment of at least 10%, at least 20%, at least 30%, at least 40%, at least 50%, at least 60%, at least 70%, at least 80% or at least 90%, compared to the target prior to treatment, or compared to like, untreated targets.

In an certain embodiments, the therapeutic effect is a reduction in the abundance of SASP cells or SASP factors in the selected compartment. The reduction in SASP cells or SASP factors may be any reduction in the absolute number of SASP cells or factors in the selected compartment, for example, a reduction of at least 10%, at least 20%, at least 30%, at least 40%, at least 50%, at least 60%, at least 70%, at least 80% or at least 90%, compared to the target prior to treatment or compared to like, untreated targets.

In another embodiment, immune infiltration is a major driver of disease and disease progression, and the therapeutic effect is a reduction in immune infiltration, for example, a reduction in the infiltration of one or more selected immune cells types, for example, T lymphocytes, B lymphocytes, dendritic cells, or macrophages, including monocytes.

In various embodiments, the therapeutic effect is defined with respect to a selected condition, wherein the condition is caused by, mediated by, exacerbated by, or is the result of an accumulation of senescent cells. With regards to a selected condition, exemplary therapeutic effects include: ameliorating one or more symptoms or morbidities of the condition, reversing one or more morbidities of the condition, curing one or more symptoms or morbidities of the condition, slowing the progression of the condition, halting the progression of the condition, or reducing the pathological processes that underlie the condition. Treatment, as used herein, will also encompass preventative treatment. With regards to a selected condition, exemplary preventative therapeutic effects include, for example, preventing the onset of the condition, reducing the probability of the condition manifesting, ameliorating the physiological processes that promote or cause the condition, or otherwise reducing the risk, severity, or progression of the condition.

Senescence-Associated Conditions. In various embodiments, the senescence associated condition is an inflammatory or autoimmune condition. For example, in various embodiments, the an inflammatory or autoimmune condition is selected from the group consisting of acute and chronic lung inflammation, lung fibrosis (including idiopathic lung fibrosis), ankylosing spondylarthritis, arthritis (including psoriatic arthritis, osteoarthritis, reactive arthritis, juvenile, and rheumatoid arthritis), arthropathy, asthma, Ataxia Telangiestasi, cachexia, chronic bronchitis, chronic pulmonary obstructive diseases, Crohn's disease, cystic fibrosis, Ehlers-Danlos Syndrome, endotoxic shock, fibromyalgia, gout, hypermobility syndrome, inflammatory bowel disease (Crohn's disease and ulcerative colitis), ischemia induced inflammation, ischemia-reperfusion injury, kidney ischemia, kyphosis, limb ischemia, liver fibrosis, local and systemic inflammation, lupus, metabolic acidosis, multiple sclerosis, osteoarthritis, psoriasis, sarcopenia, scleroderma and vasculitis, sepsis or septic shock, and ulcerative colitis.

In various embodiments, the senescence associated condition is a condition caused by metabolic dysfunction associated with the accumulation of senescent cells. For example, in various embodiments, the condition is metabolic dysfunction associated with the accumulation of senescent preadipocytes in epidydmal white adipose tissue, for example, as occurs in Type II diabetes.

In some embodiments, the senescence associated condition is diabetes or a diabetes-related condition. For example, in various embodiments, the condition is diabetes Type I, diabetes Type II diabetes-associated inflammation, diabetic ulcer, diabetic nephropathy, or diabetic adhesive capsulitis, metabolic dysfunction associated with the accumulation of senescent preadipocytes in epidydmal white adipose tissue, glucose intolerance, glucose dysregulation and other symptoms of diabetes.

In some embodiments, the senescence associated condition is a cardiovascular condition. For example, in various embodiments, the senescence associated condition is a cardiovascular condition selected from the group consisting of angina, aortic aneurysm, arrhythmia, atherosclerosis, brain aneurysm, cardiac diastolic dysfunction, cardiac fibrosis, cardiac stress resistance, cardiomyopathy, carotid artery disease, chronic obstructive pulmonary disease, congestive heart failure, coronary artery disease, coronary thrombosis, endocarditis, hypertension, hypercholesterolemia, hyperlipidemia, idiopathic pulmonary fibrosis, mitral valve prolapse, myocardial infarction, peripheral vascular disease, and stroke.

In some embodiments, the senescence associated condition is a neurodegenerative condition. For example, in various embodiments, the senescence associated condition is a neurodegenerative condition selected from the group consisting of Alzheimer's disease, Parkinson's disease, multiple sclerosis, amyotrophic lateral sclerosis, Huntington's disease, epilepsy, chronic traumatic encephalopathy, frontotemporal dementia, dementia, and motor neuron dysfunction.

In various embodiments, the senescence associated condition is an age-related condition. For example, the age-related condition may be a condition selected from the group consisting of loss of pulmonary function, macular degeneration, renal failure, frailty, muscle fatigue, liver fibrosis, pancreatic fibrosis, oral submucosa fibrosis, progressive muscle loss, decreased bone-density, age-associated memory impairment, age-related hearing loss, sarcopenia, skin atrophy, brain atrophy, arteriosclerosis, pulmonary emphysema, immunologic incompetence, cataracts, skin wrinkling, and graying of hair.

### Exemplary Embodiments

In one implementation, the invention encompasses an agent for use in a method of reducing the number of senescent cells in a selected target tissue, organ, or compartment of a subject;

wherein the agent may comprise any of: an activator of iNKT cells; an agent which promotes the removal of senescent cells by direct or indirect iNKT cell activity; a glycolipid activator of iNKT cells; GalCer; a GalCer derivative; a glycolipid activator of iNKT cells complexed with CD1d protein, including a CD1d dimer or CD1d tetramer; GalCer complexed with CD1d protein, including a CD1d dimer or CD1d tetramer; an antibody that binds to the TCR of iNKT cells; a cell expressing CD1d that is functionalized or loaded with a glycolipid activator of iNKT cells; a dendritic cell functionalized or loaded with a glycolipid activator of iNKT cells; a cell expressing CD1d that is functionalized or loaded with GalCer or a GalCer derivative; a dendritic cell functionalized or loaded with a GalCer or a GalCer derivative; an iNKT cell; an autologous iNKT cell expanded ex vivo; a iNKT precursor; or an autologous iNKT cell precursor;
and
wherein the method encompasses administering the one or more iNKT agents to the subject in a therapeutically effective amount.

In some implementations, the agent is delivered directly or locally to the target tissue, organ, or compartment. In some implementations, the target tissue, organ, or compartment of a subject comprises any of: peripheral blood, the lymph system, the brain, kidney, liver, pancreas, lung; adipose tissue; white adipose tissue; brown adipose tissue; skeletal muscle; heart; pancreas; kidney; intestine; colon; hypothalamus; cardiac tissue; liver; bladder, spleen; lymph node; dermis; stomach; lung; pancreas, brain; ocular tissue; auditory canal or cells the ear such as hair cells; spinal cord; heart, esophagus, sinus tissues; testis, ovary, bone, peripheral nerve, cartilage, soft tissue, an element of the circulatory system, hair follicles, epidermis, reproductive organs; digestive tract, bladder, airway, or the entire subject.

In another implementation, the scope of the invention encompasses an agent for use in a method of treating a senescence-associated condition in a subject;

wherein the agent may comprise any of: an activator of iNKT cells; an agent which promotes the removal of senescent cells by direct or indirect iNKT cell activity; a glycolipid activator of iNKT cells; GalCer; a GalCer derivative; a glycolipid activator of iNKT cells complexed with CD1d protein, including a CD1d dimer or CD1d tetramer; GalCer complexed with CD1d protein, including a CD1d dimer or CD1d tetramer; an antibody that binds to the TCR of iNKT cells; a cell expressing CD1d that is functionalized or loaded with a glycolipid activator of iNKT cells; a dendritic cell functionalized or loaded with a glycolipid activator of iNKT cells; a cell expressing CD1d that is functionalized or loaded with GalCer or a GalCer derivative; a dendritic cell functionalized or loaded with a GalCer or a GalCer derivative; an iNKT cell; an autologous iNKT cell expanded ex vivo; a iNKT precursor; or an autologous iNKT cell precursor; and
wherein the condition is one or more of: an inflammatory condition; an autoimmune condition; acute or chronic lung inflammation, lung fibrosis; idiopathic lung fibrosis, ankylosing spondylarthritis, arthritis; psoriatic arthritis; osteoarthritis; reactive arthritis; juvenile arthritis; rheumatoid arthritis; arthropathy; asthma; Ataxia Telangiestasi; cachexia; chronic bronchitis; chronic pulmonary obstructive diseases; Crohn's disease; cystic fibrosis; Ehlers-Danlos Syndrome; endotoxic shock; fibromyalgia; gout; hypermobility syndrome; inflammatory bowel disease; Crohn's disease; ulcerative colitis; ischemia induced inflammation; ischemia-reperfusion injury; kidney ischemia; kyphosis; limb ischemia; liver fibrosis; local or systemic inflammation; lupus; metabolic acidosis; multiple sclerosis; osteoarthritis; psoriasis; sarcopenia; scleroderma; vasculitis; sepsis or septic shock; a condition caused by metabolic dysfunction associated with the accumulation of senescent cells; metabolic dysfunction associated with the accumulation of senescent preadipocytes in epidydmal white adipose tissue; diabetes or a diabetes-related condition; Type I diabetes; Type II diabetes; Type II diabetes—associated inflammation; diabetic ulcer; diabetic nephropathy; diabetic adhesive capsulitis; glucose intolerance; glucose dysregulation; a cardiovascular condition; angina; aortic aneurysm; arrhythmia; atherosclerosis; brain aneurysm; cardiac diastolic dysfunction; cardiac fibrosis; cardiac stress resistance; cardiomyopathy; carotid artery disease; chronic obstructive pulmonary disease; congestive heart failure; coronary artery disease; coronary thrombosis; endocarditis; hypertension; hypercholesterolemia; hyperlipidemia; idiopathic pulmonary fibrosis; mitral valve prolapse; myocardial infarction; peripheral vascular disease; stroke; a neurodegenerative condition; Alzheimer's disease; Parkinson's disease; multiple sclerosis; amyotrophic lateral sclerosis; Huntington's disease; epilepsy; chronic traumatic encephalopathy; frontotemporal dementia; dementia; motor neuron dysfunction; an age-related condition; loss of pulmonary function; macular degeneration; renal failure; frailty; muscle fatigue; liver fibrosis; pancreatic fibrosis; oral submucosa fibrosis; progressive muscle loss; decreased bone-density; age-associated memory impairment; age-related hearing loss; sarcopenia; skin atrophy; brain atrophy; arteriosclerosis; pulmonary emphysema; immunologic incompetence; cataracts; skin wrinkling; and graying of hair;
wherein the method encompasses administering agent to the subject in a therapeutically effective amount.

In another embodiment, the invention encompasses an agent for use in a method of reducing a pathological process in a target tissue, organ, or compartment, or an agent for use in a method of reducing inflammation associated with a senescence-associated condition;

wherein the agent may comprise any of: an activator of iNKT cells; an agent which promotes the removal of senescent cells by direct or indirect iNKT cell activity; a glycolipid activator of iNKT cells; GalCer; a GalCer derivative; a glycolipid activator of iNKT cells complexed with CD1d protein, including a CD1d dimer or CD1d tetramer; GalCer complexed with CD1d protein, including a CD1d dimer or CD1d tetramer; an antibody that binds to the TCR of iNKT cells; a cell expressing CD1d that is functionalized or loaded with a glycolipid activator of iNKT cells; a dendritic cell functionalized or loaded with a glycolipid activator of iNKT cells; a cell expressing CD1d that is functionalized or loaded with GalCer or a GalCer derivative; a dendritic cell functionalized or loaded with a GalCer or a GalCer derivative; an iNKT cell; an autologous iNKT cell expanded ex vivo; a iNKT precursor; or an autologous iNKT cell precursor; and
wherein the method encompasses administering the iNKT agent to the subject in a therapeutically effective amount.

In some implementations, the pathological process is inflammation. In some implementations, the inflammation is inflammation associated with the accumulation of senescent cells exhibiting SASP. In some implementations, the pathological process is fibrosis. In some implementations, the agent is delivered directly or locally to the target tissue, organ, or compartment. In some implementations, the target tissue, organ, or compartment of a subject comprises any of: peripheral blood, the lymph system, the brain, kidney, liver, pancreas, lung; adipose tissue; white adipose tissue; brown adipose tissue; skeletal muscle; heart; pancreas; kidney; intestine; colon; hypothalamus; cardiac tissue; liver; bladder, spleen; lymph node; dermis; stomach; lung; pancreas, brain; ocular tissue; auditory canal or cells the ear such as hair cells; spinal cord; heart, esophagus, sinus tissues; testis, ovary, bone, peripheral nerve, cartilage, soft tissue, an element of the circulatory system, hair follicles, epidermis, reproductive organs; digestive tract, bladder, airway, or the entire subject.

In another implementation, the invention encompasses an iNKT activator or iNKT cell or precursor as a senolytic agent, wherein the iNKT activator or iNKT cell promotes the removal of senescent cells by the direct and/or indirect activity of iNKT cells;

wherein the agent may comprise any of: an activator of iNKT cells; a glycolipid activator of iNKT cells; GalCer; a GalCer derivative; a glycolipid activator of iNKT cells complexed with CD1d protein, including a CD1d dimer or CD1d tetramer; GalCer complexed with CD1d protein, including a CD1d dimer or CD1d tetramer; an antibody that binds to the TCR of iNKT cells; a cell expressing CD1d that is functionalized or loaded with a glycolipid activator of iNKT cells; a dendritic cell functionalized or loaded with a glycolipid activator of iNKT cells; a cell expressing CD1d that is functionalized or loaded with GalCer or a GalCer derivative; a dendritic cell functionalized or loaded with a GalCer or a GalCer derivative; an iNKT cell; an autologous iNKT cell expanded ex vivo; a iNKT precursor; or an autologous iNKT cell precursor.

### EXAMPLES

### Example 1. Invariant Natural Killer T Cells Coordinate Removal of Senescent Cells

Introduction. The accumulation of senescent cells within tissues can drive the progression of diseases. While removal of senescent cells with senolytic drugs has emerged as a promising therapeutic approach, the ubiquitous target of these drugs makes clinical applications challenging. In healthy tissue, endogenous immune surveillance mechanisms limit the build-up of senescent cells by targeted removal of senescent cells. The failure of the immune surveillance to efficiently recognize and target senescent cell clearance could result in the accumulation of senescent cells. The identity of the endogenous immune surveillance that mediated senescent cell clearance in vivo is not clear.

To address this question, tissues that showed accumulation of senescent cells in vivo were studied. Senescent preadipocytes have been reported to accumulate in the white adipose tissue (WAT) of both mice chronically fed a high fat diet (HFD) and in obese humans. Genetic ablation (of p16^{Ink4a}-expressing cells) or use of senolytic compounds resulted in the elimination of senescent preadipocytes, suggesting that HFD mice could be used as a model to investigate immune surveillance in senescent preadipocyte clearance. To identify mechanisms mediating immune surveillance of such senescent cells in the WAT, upregulated cell surface receptors that could mediate interactions between senescent cells and immune cells were searched for. Using single-cell analysis, the inventor of the present disclosure previously showed that mRNA encoding beta-2-microglobulin (B2M), a component of the class I major histocompatibility complex (MHC), is highly upregulated in senescent beta cells, leading to the present exploration of class I MHC-like molecules among preadipocytes from the WAT of mice. An MHC Class I-like molecule CD1d was identified, that is normally expressed by antigen-presenting cells but showed elevated expression specifically in the senescent preadipocytes suggesting a potential link between senescence and immune regulation.

CD1d in complex with B2M presents lipid antigens to a class of T lymphocytes known as invariant Natural Killer T (iNKT) cells. iNKT cells have an invariant T cell receptor (TCR) and are readily identified in both humans and mice with antigen-loaded CD1d tetramers, making this a population of T cells that has been comprehensively surveyed. iNKT cells decline in frequency and function in humans with age, but a link between this decrease and the increase in senescent cells during aging or disease has not been hypothesized or investigated. Herein is described how lipid antigen presentation to activate iNKT cells can constitute an endogenous surveillance system that can be manipulated to clear senescent cells in disease models.

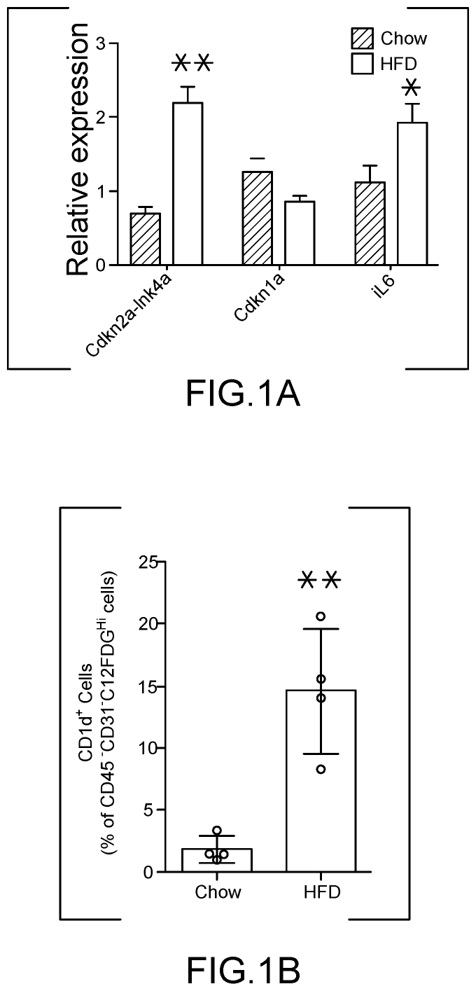

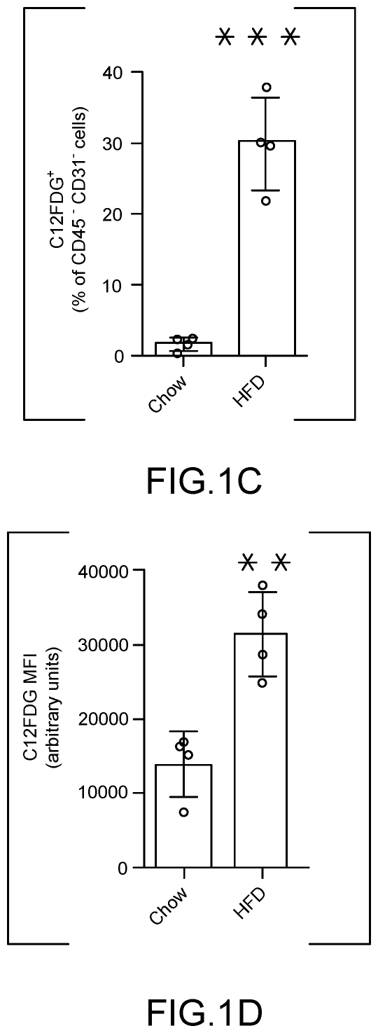

Results. Senescent cells accumulate in eWAT of HFD mice. To characterize obesity-associated senescent cells in the epididymal WAT (eWAT), male mice were fed a HFD (60% kcal from fat) for 16 weeks (from 6 weeks until 22 weeks of age) and then isolated the stromal vascular fraction (SVF) associated with the eWAT, which is comprised predominantly of preadipocytes (˜75%), but also includes mesenchymal stem cells and endothelial cells along with resident immune cells. The immune cells were depleted from SVF using CD45 antibody-labeled nanobeads prior to probing for senescent cells in both chow-fed (control) and HFD-fed mice. Senescence-associated-βgal (SA-βgal) activity was measured and the mRNA levels of genes associated with senescence and the senescence-associated secretory phenotype (SASP) in the remaining SVF cells. This preadipocyte-enriched population, when isolated from obese mice, consistently showed increased SA-βgal staining and elevated expression of several senescence (Cdkn2a^{Ink4a}, Il6) genes, whereas those from age-matched control mice did not (FIG. 1A). This was similar to when comparing proliferative and senescent preadipocytes. Consistent with the upregulation of CD1d on senescent preadipocytes in vitro, CD1d expression was increased in preadipocytes isolated from HFD fed mice compared to chow mice (FIG. 1B).

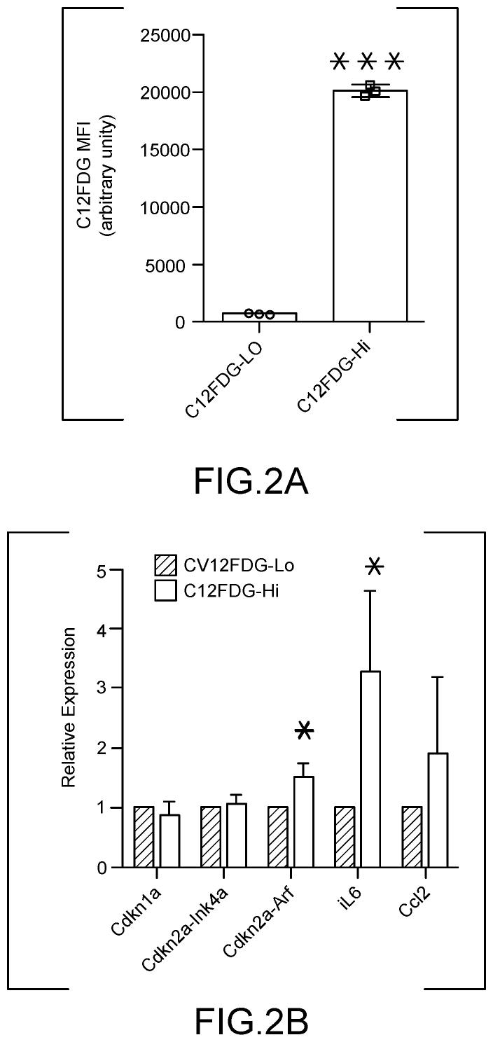

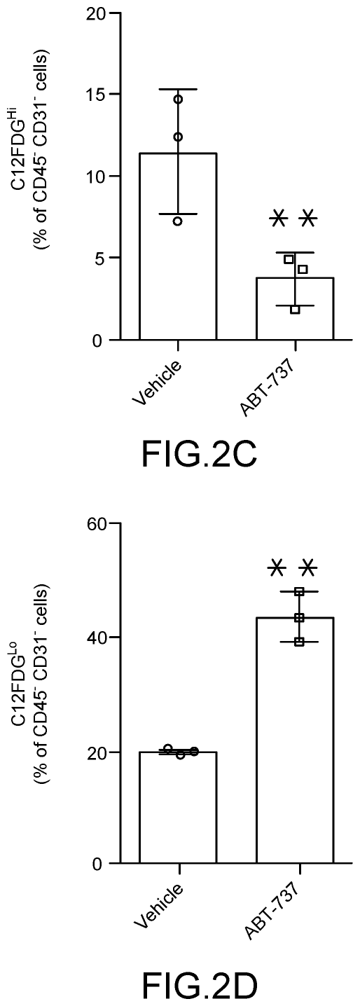

C₁₂FDG^{Hi} cells mark secretory senescent cells that are sensitive to senolytics. To quantify the number of senescent preadipocytes from the eWAT of obese vs. lean mice, a common flow cytometric assay that measures SA-βgal activity with the fluorogenic substrate C₁₂FDG was used, ad described in Debacq-Chainiaux, et al., 2009. Protocols to detect senescence-associated beta-galactosidase (SA-betagal) activity, a biomarker of senescent cells in culture and in vivo. Nat. Protoc. 4, 1798-806. Based on median fluorescence intensity of C₁₂FDG, a subpopulation of CD45⁻ CD31⁻ preadipocytes was identified from the SVF of HFD mice that had elevated SA-βgal activity when compared to those from control diet-fed counterparts. The CD31⁺ cells did not appear to contribute significantly to the overall C₁₂FDG signal, and subsequently, CD45⁻ negative gating was used to characterize the senescent cells (FIGS. 1C and 1D). Next, it was explored whether SA-βgal activity levels could distinguish different subsets of senescent cells, FACS was used to separate preadipocytes from HFD fed mice and separated the high fluorescence (highest ˜25%, C₁₂FDG^{Hi}) from those with low fluorescence (lowest ˜20%, C₁₂FDG^{Lo}) such that the levels of C₁₂FDG fluorescence were ˜30-fold higher in the C₁₂FDG^{Hi} vs. C₁₂FDG^{Lo} population (FIG. 2A). While Cdkn1a and Cdkn2a^{Ink4a} were expressed at the same levels in both these preadipocyte subsets, C₁₂FDG^{Hi} cells had elevated mRNA levels of secretory genes that comprise SASP such as Il6 and Ccl2 relative to C₁₂FDG^{Lo} cells from the same obese mice (FIG. 2B). the C₁₂FDG^{Lo} cells were designated as replicative senescent and C₁₂FDG^{Hi} as secretory senescent cells.

Next, it was tested whether these C₁₂FDG^{Hi} preadipocytes could be ablated with senolytic drugs. The Bcl2 family is known to be upregulated in senescent cells, and ABT-737, a BH3 mimetic that is well-tolerated in vivo, acts as a senolytic by inducing apoptosis preferentially in senescent cells²¹. HFD-fed and control mice were treated with ABT-737, and C₁₂FDG^{Hi} preadipocytes from the SVF of their eWAT were quantified. Treatment with ABT-737 reduced the number of C₁₂FDG^{Hi} cells and concordantly increased the relative number of C₁₂FDG^{Lo} cells, within the preadipocyte-enriched population of the SVF from the eWAT of HFD-fed mice when compared to vehicle-treated controls (FIG. 2C).

Alpha-GalCer treatment results in activation and expansion of iNKT in eWAT. Next, it was considered whether iNKT cells could similarly regulate the numbers of secretory senescent cells within the WAT of obese mice. First, the number of tissue-resident iNKT cells in the eWAT of both chow- and HFD-fed mice was quantified using a CD1d tetramer loaded with alpha-GalCer to specifically detect these cells. Using this approach, it was found that the number of iNKT cells normalized to total live SVF cells within the eWAT was consistently reduced in the context of diet-induced obesity. Treatment of HFD mice with alpha-Galactosylceramide (GalCer), a well-known lipid antigen that specifically activates iNKT cells when presented on CD1d in vitro and in vivo was sufficient to dramatically increase iNKT cell numbers compared with similarly obese mice treated with vehicle. This finding is consistent with the activation of iNKT cells in the eWAT by GalCer treatment.

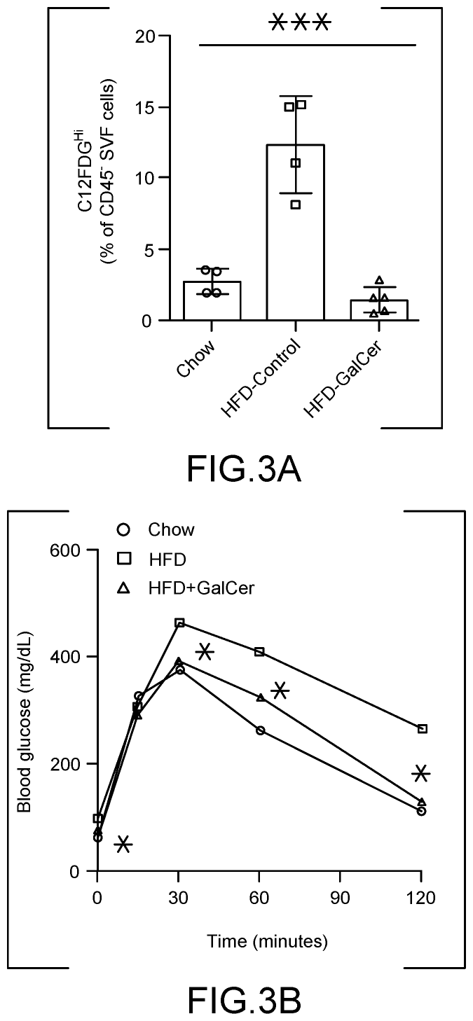

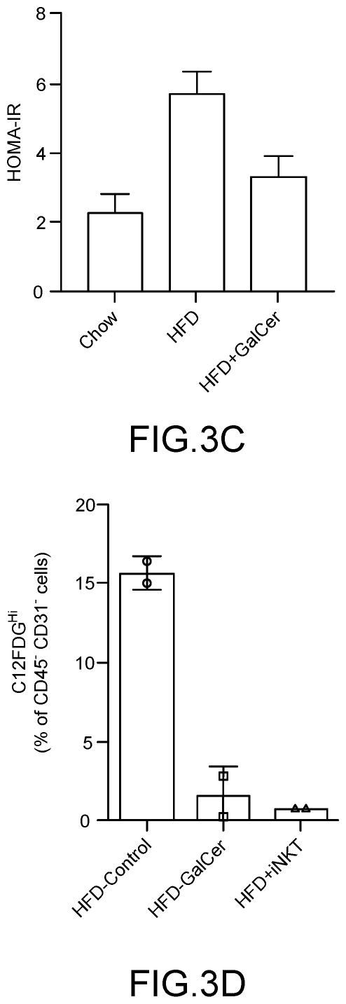

GalCer treatment of HFD mice leads to removal of senescent cells and normalizes blood glucose. To explore whether activation of iNKT regulates senescent cells, mice were treated with GalCer to assess the senescent cells in the SVF fraction of eWAT. HFD mice were injected with either GalCer or vehicle and SA-βgal activity among eWAT-derived SVF cells by FACS for C₁₂FDG was analyzed. As expected, mice with diet-induced obesity injected with the vehicle, had an increased frequency of C₁₂FDG^{Hi} preadipocytes in the eWAT relative to that seen in lean mice fed the control diet. By contrast, treating obese mice with GalCer was remarkable in its ability to deplete the number of these C₁₂FDG^{Hi} cells to levels akin to those seen in lean control mice (FIG. 3A). It was also tested whether the GalCer-induced activation of iNKT cells was required for the removal of senescent cells. We used Traj18-deficient mice that lack the invariant chain of the T cell receptor³⁹. HFD fed WT and Traj18^{-/-} mice were injected with GalCer and C12FDG+ SVF cells were used to assess senescent cells. -GalCer treatment resulted in reduction of senescent cells in WT HFD mice but failed to reduce senescent cells in Traj18^{-/-} mice. To assess the effect of eliminating senescent cells on metabolic health, blood glucose and insulin levels were monitored in HFD-fed mice treated with GalCer. HFD-fed mice that received GalCer had significantly lower fasting glucose, improved glucose tolerance, and improved insulin sensitivity compared to vehicle-treated HFD-fed mice and resembled chow-fed mice (FIG. 3B).

Adoptive transfer of activated iNKT leads to reduced senescent preadipocytes in HFD mice. To confirm that the clearance of WAT senescent cells in response to in vivo -GalCer delivery was mediated by activated iNKT cells as opposed to any off-target effects, the ability of adoptively transferred eWAT-isolated iNKT cells to clear senescent cells from the eWAT of recipient mice rendered obese by prior HFD feeding was assessed. iNKT cells were FACS-isolated from the eWAT of GalCer-treated HFD mice using the CD1d-loaded tetramer, and ˜150-300,000 of these iNKT cells were transferred into recipient mice in two separate experiments.

Remarkably, such transfer was sufficient to markedly deplete the number of C₁₂FDG^{Hi} preadipocytes otherwise present in the eWAT of HFD-fed control recipient mice relative to untreated controls. Notably, the effect of adoptive transfer phenocopied that of treating HFD-fed mice with GalCer directly, which was used as a positive control for these experiments (FIG. 3D). Together these data directly implicate the involvement of activated resident eWAT iNKT cells in the clearance of senescent preadipocytes in a model where they accumulate in the WAT.

Activation of iNKT in an IPF model results in decreased senescent cells and reduced fibrosis. To address the generality of iNKT-mediated clearance of senescent cells and whether such mechanisms operate in other disease settings, bleomycin-induced injury in the lung was investigated, a model for interstitial lung disease. Intratracheal instillation of the chemotherapeutic agent bleomycin induces epithelial damage, followed by infiltration of inflammatory cells into the lung interstitium and alveolar space. Previous studies have demonstrated that senescent epithelial and mesenchymal cells accumulate after bleomycin-induced injury and that senolytic treatment can clear these senescent cells. Accumulation of senescent epithelial cells also occurs in lung tissue of patients with idiopathic lung fibrosis (IPF), and senolytic treatment in an open-label study of IPF patients showed benefit suggesting that the bleomycin-induced injury is a good model for senolytic approaches to lung fibrosis.

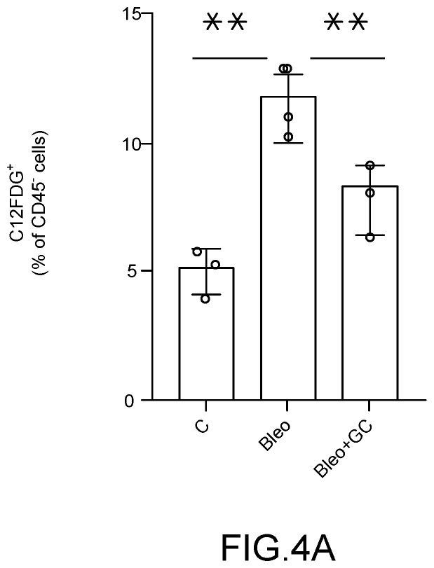

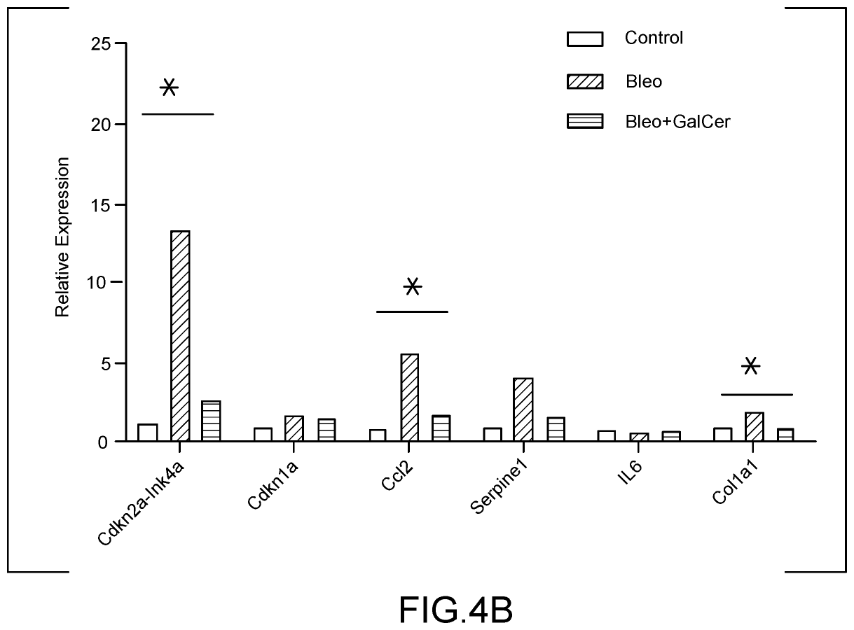

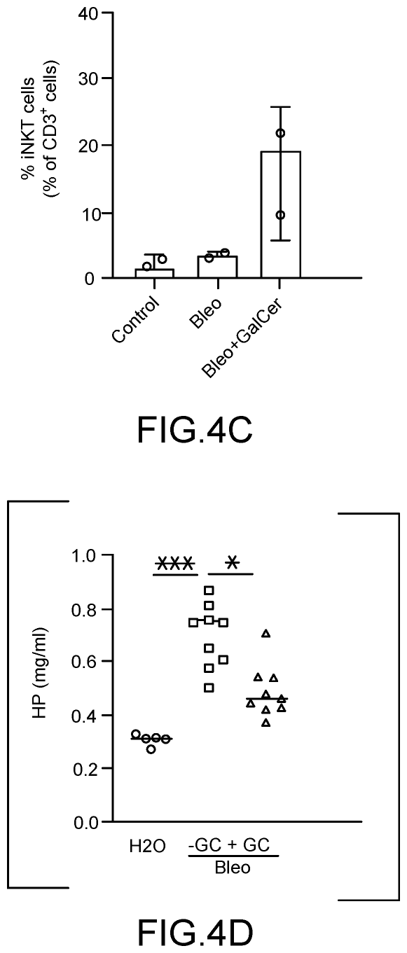

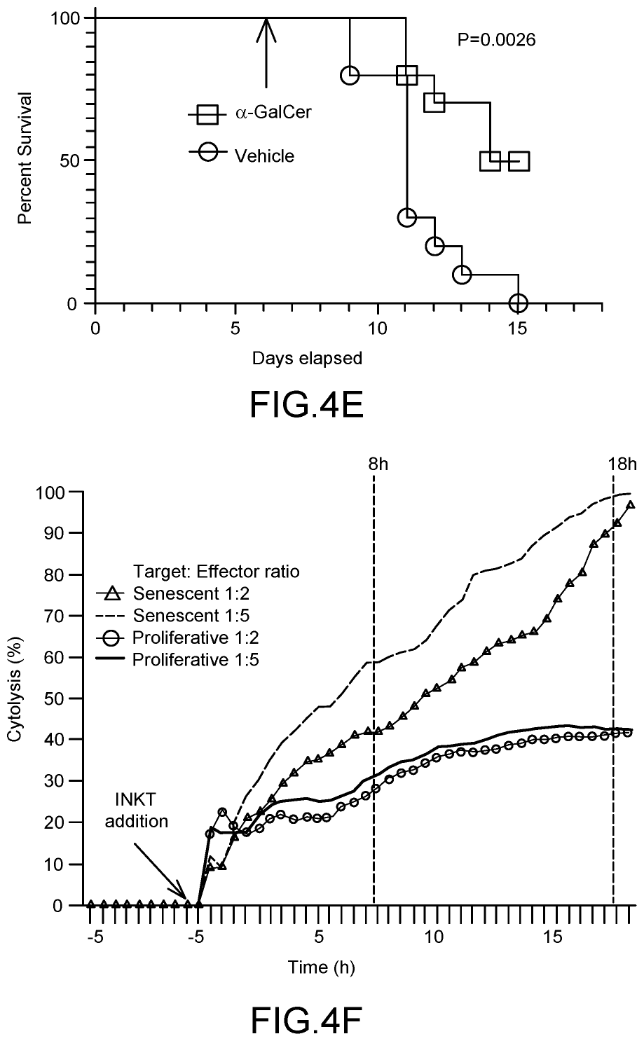

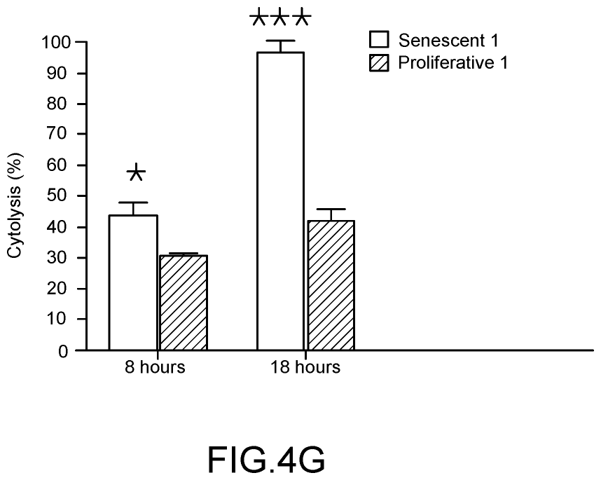

To test whether iNKT cells could eliminate senescent cells in bleomycin-induced lung injury model, bleomycin-induced injury was incurred in a cohort of C57BL6 male mice. Ten days after injury, these mice were either treated systemically with GalCer or left untreated, while age-matched uninjured mice treated with the vehicle on day 10 served as controls. Lungs were harvested 14 days after injury, and the proportion of senescent epithelial and mesenchymal cells was quantified, as was the relative number of iNKT cells. As seen in WAT-associated preadipocytes from HFD-fed mice, bleomycin-induced lung injury induced a marked increase in the number of senescent CD45⁻ lung cells, as analyzed by quantifying SA-βgal activity using the C₁₂FDG assay (FIG. 4A). Importantly GalCer treatment of bleomycin-injured mice decreased the number of such CD45⁻ C₁₂FDG⁺ lung cells to levels on par with those in the lungs of uninjured control mice. As with previous studies, both epithelial and mesenchymal senescent cells were affected by GalCer treatment. To confirm that the changes in SA-βgal activity reflected senescent cells, mRNA levels of senescence and SASP markers was analyzed. Consistent with the C₁₂FDG assay, CD45⁻ lung cells from bleomycin-injured mice had increased mRNA levels of senescence (Cdkn2a^{Ink4a}) and SASP (Ccl2, Serpine1) markers. Moreover, GalCer treatment of bleomycin-injured mice reduced mRNA levels of these marker genes, consistent with the diminished SA-βgal activity we observed using the C₁₂FDG assay (FIG. 4B). As in the eWAT, GalCer treatment also induced expansion of lung parenchymal iNKT cells in the context of bleomycin-induced injury (FIG. 4C). To address whether this iNKT expansion and its associated senolytic effect also suppressed lung fibrosis, the hydroxyproline (HP) assay was used to measure whole lung collagen content in bleomycin-injured mice treated with or without GalCer. Importantly, -GalCer treatment of lung-injured mice significantly reduced lung hydroxyproline content, consistent with suppressed fibrosis (FIG. 4D). Removal of senescence and suppression of fibrosis has beneficial effects on survival as mortality of GalCer-injected mice was significantly reduced compared to the vehicle-injected mice (FIG. 4E). These findings are generally consistent with earlier work wherein GalCer and NKT cell activity were explored in the treatment of bleomycin-induced injuries, including Kimura et al., 2004. Treatment with α-Galactosylceramide Attenuates the Development of Bleomycin-Induced Pulmonary Fibrosis. J. Immunol. 172, 5782-5789 and Kim et al., 2005. Natural killer T (NKT) cells attenuate bleomycin-induced pulmonary fibrosis by producing interferon-gamma. The American journal of pathology, 167(5), 1231-1241. These early studies were more preventative rather than therapeutic as either GalCer or adoptively transferred iNKT cells was administered prior to bleomycin injury, potentially mitigating the development of lung injury rather than reversing fibrosis after it was established. Importantly, these prior studies did not evaluate or link their observed effects to a reduction of senescent cells. Taken together, these results suggest that iNKT cell activation within the lung by GalCer treatment attenuates the accumulation of senescent cells during bleomycin-induced IPF, producing an anti-fibrotic effect and improved survival.

Activated iNKT cells are preferentially cytotoxic to human senescent cells. To address whether iNKT cells can directly act on senescent cells in humans, an in vitro cytotoxicity assay was developed. Peripheral blood mononuclear cells (PBMCs) from a healthy human donor were used to both generate dendritic cells (DCs) and isolate iNKT cells, which were then cultured separately. In parallel, human lung fibroblasts (WI-38) were cultured and treated with etoposide to induce senescence. Untreated proliferating WI-38 cells were used as controls. -GalCer-loaded DCs were used to activate and expand iNKT cells. Activated iNKTs were combined with either senescent or proliferative (untreated) WI-38 cells in co-cultures and employed the xCELLigence™ platform (Agilent Systems, Inc.) to measure cellular impedance, and thus, continuously monitor real-time kinetic behavior indicative of cell number and attachment. The non-specific kinase inhibitor staurosporine, which induces rapid apoptosis independently of cell state, was used as a positive control for cytotoxicity (data not shown). Senescent or proliferating WI-38 cells were seeded into wells, and activated iNKT cells were added to these wells at different target-to-effector ratios. iNKT cells remain in suspension and do not adhere to the wells and as such contribute negligibly to impedance. Percent cytolysis was calculated and used for comparing the relative efficacy of activated iNKT towards senescent and non-senescent WI-38 target cells. Co-culture of activated iNKT cells with proliferative WI-38 cells showed minimal cell killing at two target:effector ratios. By contrast, co-culturing similarly activated iNKT cells with senescent WI-38 cells induced a significant dose and time-dependent increase in cytolysis of senescent fibroblasts, reaching a maximum of ˜100% at both target:effector ratios by 18 h (FIG. 4F). Moreover, even at the lowest target:effector ratio of 1:2, activated iNKT were significantly more cytotoxic towards senescent cells at both the midpoint (8 h) and endpoints (18 h) of the assay (FIG. 4G). These results indicate that activated primary human iNKT cells can preferentially induce cytotoxicity of senescent over proliferative cells.

Discussion Senescence phenotypes vary depending on the context and trigger, and it is apparent that different immune cell types co-operate to remove senescent cells in different tissues. For instance, NK cells mediate surveillance of senescent activated stellate cells in the liver, and recent work has shown that the perforin-granzyme pathway is vital for cytotoxic effector function in the elimination of senescent cells in multiple tissues during aging and chemically induced liver fibrosis. A recent paper leveraged the expression of a urokinase-type plasminogen activator receptor (uPAR) on oncogene-induced senescent cells to develop antigen-specific CAR-T cells that could ablate the senescent population. This approach to senolysis may be well suited to situations where senescent cells express an aberrant antigen in a particular tissue context, such as in oncogene-induced senescence associated with cancer, but not in chronic diseases associated with DNA damage-induced senescence as uPAR was not identified on senescent preadipocytes in the HFD mice. the reliance on antigen specificity was overcome by using iNKT cells here as the means to achieve effective senolysis, and by doing so could clear senescent cells of different types in different tissue environments despite a common delivery method. iNKT cells have both adaptive and innate features that make them uniquely suited to coordinating an early response to the elimination of aberrant cells, such as virus-infected cells or senescent cells. Activation of iNKT cells with GalCer has been found to reverse adverse metabolic phenotypes in the HFD mouse model (for example, as disclosed in Lynch et al., 2012, Schipper et al., 2012, and Ji et al., 2012) as well as the fibrosis induced by lung injury (as described in Kimura et al., 2004 and Kim et al., 2005). The results presented herein provide the first evidence that iNKT cells can eliminate senescent cells in these two distinct models where tissue dysfunction is associated with the accumulation of senescent cells.

While senolytics have been the dominant approach to move senescence-clearing therapies to the clinic, several reasons make the activation of iNKT cells a more promising approach. Senolytics target anti-apoptotic pathways that are upregulated not only in senescent cells, but these pathways can also be active in non-senescent cells. To avoid toxicity, clinical trials with senolytics are administered locally to reduce the risk of side effects. In contrast, the unique invariant TCR on iNKT cells allows exquisite specificity for lipid antigens such as α-GalCer, and the results presented herein demonstrate that iNKT cell activation by α-GalCer renders them specifically cytotoxic to senescent rather than non-senescent cells. Another advantage is that the activation of iNKTs is short-lived, avoiding an unchecked and prolonged cytolytic effect. Pre-clinical and clinical studies evaluating the safety of α-GalCer showed that it has generally been well-tolerated over a wide range of doses with minimal side effects, for example, as described in Wolf et al., 2018. Novel Approaches to Exploiting Invariant NKT Cells in Cancer Immunotherapy. Frontiers in Immunology 9, 384. As demonstrated herein, specific activation of surveillance responses by iNKT cells provides a next generation of approach to eliminate inflammatory senescent cells associated with chronic diseases.

MATERIALS AND METHODS. Mouse husbandry. All procedures involving mice were performed according to the IACUC standards following ethics approval by the animal committee at the University of California, San Francisco. Male C57BL6/J on chow or HFD fed mice. HFD mice were fed a high-fat diet (60% kcal from fat) from 6 weeks until 22 weeks of age. All experiments on HFD mice were performed at 22-24 weeks total age (16-18 weeks on HFD). Age-matched male C57BL6/J mice on standard rodent chow diet were used as controls. For the lung injury model, 10-week-old male C57BL/6J mice were used. The animals were divided into three groups: the control group, the injured group, and the α-GalCer treated group. The animals in the control group received vehicle only (i.p.) on day 10. The injured group animals received bleomycin (3 U/kg) via the intratracheal route at the start of the experiment (day 0). The animals in the α-GalCer treated groups received bleomycin (3 U/kg) on day 0, and i.p. injection of α-GalCer (2 μg/mouse) on day 10. The animals in all the groups were sacrificed on day 14, and lungs were harvested for isolation of primary pulmonary Alveolar Type II cells (ATII) on the same day. For the hydroxyproline assay, mice were euthanized 21 days after injury.

Epidydmal white adipose tissue stromal vascular fraction (eWAT SVF) isolation. eWAT SVF was isolated using standard protocols. Briefly, mice were euthanized, and epididymal fat pads were harvested, washed twice in D-PBS and minced. eWAT pads were digested with 1 U/ml Collagenase-D (Roche) in 1×HBSS containing 1% BSA (Sigma-Aldrich) for 1 hr at 37° C. Digested fat tissue was then strained twice through 100 μm strainers and neutralized with 30 ml of complete culture media (DMEM-F12 1:1, 10% FBS, 1×Penicillin-streptomycin). Samples were spun at 500 g for 5 minutes, shaken to resuspend the SVF pellet and then spun again. The resulting SVF pellet containing blood cells was lysed with ACKS buffer, washed in D-PBS and spun down. Total live SVF cells were counted using trypan blue and then used for various assays.

Isolation of primary epithelial cells from lung. Primary epithelial cells from murine lungs were isolated as per the method described by Sinah and Lowell³⁶ with slight modifications. Briefly, mice were euthanized with an overdose of isoflurane. A bilateral thoracotomy was performed, and lungs were exsanguinated by perfusion with PBS containing 5 mM EDTA via right ventricle after snipping left atrium. Following perfusion, lungs were instilled with 50 units/mL dispase via tracheal cannulation and allowed to sit for 5 min. Lungs were excised from the thoracic cavity and digested in dispase at 37° C. for 45 min. Cells were released from the lungs by gentle teasing and collected in sterile DMEM containing DNase I (1 mg/10 mL). To get the single-cell suspension, lung homogenate was sequentially filtered through 70 μm and 40 μm cell strainers and centrifuged at 300 g for 10 mins at 4° C. RBCs in cell pellet were lysed by resuspending in 1 mL lysis buffer for 3 min and was neutralized using 10 mL of sterile DMEM medium, centrifuged and the cell pellet resuspended in 1 ml of sterile DMEM containing DNase I. Viability of the cells was analyzed using Vi-Cell XR cell viability analyzer.

CD45⁺ cell depletion of eWAT SVF. eWAT SVF, as prepared above, was depleted of CD45⁺ cells using metal-assisted cell sorting (MACS) nanobeads according to the product protocol. The CD45-depleted (unmagnetized) fraction was collected, spun down at 500 g for 5 minutes and either plated on tissue-culture treated plates for X-gal staining or used for RNA extraction. For lung cells, ATII cells (<10⁷/100 μl) were washed with buffer and were incubated with 10 μl of mouse CD45 Nanobeads for 15 mins on ice. After incubation, cells were washed with buffer, centrifuged at 300 g for 5 min, then resuspended in 3 ml of buffer in polypropylene tubes, and placed in the magnetic column for 15 mins at 4° C. After 15 mins, the 2.5 ml of supernatant was collected as the unlabeled or CD45-depleted fraction. The cells in the CD45-depleted fraction were centrifuged at 300 g for 5 min and were stored in 300 μl of rnase-inhibiting nucleic acid extraction reagent (at −80° C. until RNA extraction).

X-Gal staining for SA-βgal activity. Inguinal WAT preadipocytes or eWAT SVF from chow or HFD mice, depleted of CD45⁺ cells and plated for 24 h were stained for SA-βgal activity using X-gal with a commercial kit according to the product instructions. Images were taken at 10× or 20× on a bright-field microscope.

C₁₂FDG flow cytometry assay. eWAT SVF cells isolated and counted as described above were adjusted to 3×10⁶ cells/ml in complete culture media and incubated with 33 μM C₁₂FDG for 1 hr in a 37° C. water bath. Cells were then spun down at 500 g for 5 minutes, washed with Cell Staining buffer, and stained with anti-CD45-APC (clone 30F-11) and anti-CD31-PE-Cy7 (clone 390) each at a 1:200 dilution on ice for 30 minutes. Cells were then washed in Cell Staining buffer and resuspended in Cell Staining buffer containing 1×7-AAD. Samples were analyzed on an Attune acoustic focusing cytometer. Gates were set as follows: cells (FSC-A/SSC-A), forward scatter singlets (FSC-H/FSC-A), side-scatter singlets (SSC-H/SSC-A), live cells (7-AAD⁻), non-immune cells (CD45⁻), non-endothelial cells (CD31⁻) and analyzed for C₁₂FDG fluorescence. In some cases, CD31 was not included, as it was found that the inclusion or exclusion of this population did not significantly change the overall distribution or gating of the C₁₂FDG subpopulations. Data were analyzed with FlowJo (v. 10). For Lung, 1×10⁶ cells from each sample were used to perform the assay. The cells were incubated with 66 μm of C₁₂FDG in sterile DMEM containing DNase for 1 hour at 37° C. Following the incubation, the cells were washed with cell staining buffer, blocked with anti CD16/32 mouse monoclonal antibody (1:1000, prepared by the UCSF monoclonal antibody core), and stained with APC anti-mouse CD45 (1:400), and 7-AAD (1:1000). The gating strategy followed selected 7-AAD⁻, CD45⁻, and C12FDG⁺ cells.

Flow cytometry for CD1d. Ing or eWAT preadipocytes from CAG-Luc mice at different passages were collected and stained with anti-CD1-PE (clone 1B11) in cell staining buffer. Flow cytometry was then performed on the Attune acoustic focusing cytometer. Gates were set as follows: cells (FSC-A/SSC-A), forward scatter singlets (FSC-H/FSC-A), live cells (GFP⁺), and CD1d fluorescence.

α-GalCer activation of iNKT cells. iNKT cells were activated in vivo using α-GalCer as described in Peralbo et al., 2006. Decreased frequency and proliferative response of invariant Vα24Vβ11 natural killer T (iNKT) cells in healthy elderly. Biogerontology 7, 483-492. Briefly, HFD mice were i.p. injected with 200 μl of a 10 μg/ml α-GalCer solution (2 μg total) made up in 5.6% Sucrose, 5% Tween-20. On day 3 or 4 post-injection, mice were euthanized for various experiments, as indicated.

Flow cytometry of iNKT cells. eWAT SVF cells were stained with PBS57 (α-GalCer-analog) loaded onto CD1d-PE tetramers prepared by the NIH Tetramer Core and anti-CD3-APC. As a negative control for subtracting background staining in eWAT SVF, samples were also stained with unloaded CD1d-PE tetramer and anti-CD3-APC. Live cells were enumerated with DAPI, and flow cytometry was performed on an Attune acoustic focusing cytometer. Gates were set to identify: cells (FSC-A/SSC-A), forward scatter singlets (FSC-H/FSC-A), side-scatter singlets (SSC-H/SSC-A), live cells (DAPI⁻), and the PBS57::CD1d-PE⁺/CD3-APC⁺ subpopulation. The percent of unloaded CD1d-PE⁺/CD3-APC⁺ cells were subtracted from the loaded tetramer stained populations to eliminate the percent of background stained cells. For lung, 1×10⁶ cells from each sample were used to quantify iNKT cells. The incubation, the cells were washed with cell staining buffer, blocked with anti CD16/32 mouse monoclonal antibody (1:1000), and were stained with DAPI, Tetramer (α-GalCer loaded CD1d tetramer, NIH) labeled with PE (1:100); APC labeled anti mouse CD3 (1:400). The gating strategy followed is DAPI negative, CD3 and Tetramer double positive. The iNKT-Cell count is reported as the percentage of CD3 cells staining positive with the tetramer.

Metabolic measurements. Seventeen male HFD-fed C57BL6J mice were divided into the vehicle (n=8) and the α-GalCer treated group (n=9). Age-matched male C57BL6/J mice on a standard rodent chow diet were used as controls (n=8). A glucose tolerance test was performed 10 days after α-GalCer injection. Mice were fasted overnight (14 h) before being injecting 2 g/kg glucose intraperitoneally. Blood glucose was measured at 0, 15, 30, 60, and 120 min after glucose injection using a glucometer.

Bleomycin lung injury. For measurement of lung collagen, 3 U/kg Bleomycin was instilled intratracheally, with injection of α-GalCer or vehicle at day 10, followed by lung harvest for hydroxyproline assay at 20 days. For the survival study, 4 U/kg Bleomycin was instilled intratracheally, with injection of α-GalCer or vehicle at day 5.

Adoptive transfer of iNKT cells. HFD mice were treated with α-GalCer as described above and then on day 3 sacrificed followed by eWAT SVF isolation, iNKT cell staining, and flow cytometry were carried out as described above. Approximately 150-300,000 Live iNKT cells were sorted on a Sony SH800S flow sorter, into lymphocyte media (RPMI-1640, 10% FBS, 1×antibiotic-antimycotic). Sort purity was generally 90%. Cells were then immediately spun down (500 g for 5 minutes) reconstituted in 150-200 μl D-PBS and i.p. injected into recipient HFD mice. Alongside the adoptive transfer, HFD mice were left untreated as negative controls or were treated with α-GalCer as above, to serve as positive controls. On day 4 post-transfer, HFD mice were euthanized, and C₁₂FDG assays were carried out on the eWAT SVF.

Flow sorting of C₁₂FDG^{Hi} and C₁₂FDG^{Lo} cells from eWAT SVF.eWAT SVF was isolated from HFD mice and stained using the C₁₂FDG staining procedure with anti-CD45-APC (clone 30F-11) to gate out immune cells and DAPI to identify live cells. Samples were sorted cell sorter. Gates were set to identify: cells (FSC-A/SSC-A), forward scatter singlets (FSC-W/FSC-A), side-scatter singlets (SSC-W/SSC-A), live cells (DAPI⁻), non-immune cells (CD45⁻) for C₁₂FDG fluorescence. The C₁₂FDG histogram plot was then gated on the top ˜25% highest fluorescent subpopulation (C₁₂FDG^{Hi}) and the lowest ˜20% (C₁₂FDG^{Lo}) and sorted into two different tubes containing RNA extraction reagent.

Hydroxyproline Assay. Hydroxyproline assay was performed as have been described in the art. Briefly, snap-frozen lung samples were homogenized and mixed with 50% Trichloroacetic acid (Sigma) before incubating them overnight in 12 M HCl at 110° C. The following day, the samples were reconstituted with water for 2 h. After reconstitution, they were mixed with 1.4% chloramine in 10% isopropanol and 0.5 M Sodium acetate. Finally, they were mixed with Ehrlich's solution and incubated at 65° C. for 15 mins. Absorbance was measured at 550 nm and the values were computed from the standard curve.

RNA extraction and Quantitative reverse transcriptase PCR.RNA was extracted with RNA extraction reagent using the commercial total RNA micro-prep kit according to the product instructions. Total RNA was quantified by nanovolume photospectrometer and fluorometer. Approximately 200-300 ng of total RNA was used for cDNA synthesis using a commercial cDNA synthesis kit. cDNA (250 ng) was diluted 1:5 before amplification with SYBR reagent using a real-time PCR system. Quantification was performed using with the delta-delta C_{T} method, using Cyclophilin A (Ppia) as a housekeeping gene.

In vitro cytotoxicity assay with human peripheral blood iNKT cells. Human peripheral blood mononuclear cell (PBMC) iNKT isolation and expansion were based on methods, as described in Li et al., 2013. Generation of Human iNKT Cell Lines. Bio-protocol 3 (6): e418. DOI: 10.21769/BioProtoc.418. PBMCs were isolated from healthy patients using a commercial PBMC isolation system. The isolation of CD14⁺ and CD14⁻ cells was performed using a human CD14 positive selection kit. CD14⁺ and CD14⁻ cells were cultured with recombinant proteins: 20 ng/mL GM-CSF and 20 ng/mL IL-4, or 10 U/mL IL-2 respectively. Basal media consisted of RPMI-1640 containing 1% sodium pyruvate, 1% non-essential amino acid solution, 1% L-glutamine, 1% antibiotic antimycotic, 1% HEPES, and 10% fetal bovine serum (complete media). On day five, the CD14⁺ cells were treated with 0.05 mg/mL of mitomycin C at 5×10⁶ cells/mL for 30 min. at 37° C., before being washed 3 times with PBS. Cells were then loaded with 200 ng/mL α-GalCer for one hour. iNKT cells were isolated from the CD14⁻ cell population using the anti-iNKT microbeads. The α-GalCer-loaded dendritic cells and iNKTs were co-cultured at a ratio of 5:1. Half media changes were performed every other day with complete media supplemented with 20 U/mL IL-2. On day 12, cells were stained with anti-Vα24 and anti-Vβ11 and sorted for double-positive cells on a FACS Aria II cell sorter. The isolated iNKTs recovered for 24 hours in complete media containing 20 U/ml of IL-2. WI-38 human lung fibroblasts (ATCC) were cultured to 70% confluence in media containing Eagle's Minimum Essential Medium with 1% antibiotic antimycotic and 10% FBS (maintenance media). 100 μM of etoposide was added to the media for 48 hours to induce senescence. Maintenance media was changed every other day for 6 days. 5 μg/ml of fibronectin was coated on each well of an 96 well plate for 20 min. On the seventh day post-senescence induction, proliferating control and etoposide-treated senescent WI-38 cells were seeded onto the plate at 5000 cells/well. Cells were incubated with 100 μl/well of maintenance media for 1 hour at 25° C. before going into the xCELLigence™ (Agilent Inc.) cradle. Twenty-four hours later, iNKT cells in complete media+20 U/ml of IL-2 were added at ratios of 2:1 (10,000 cells) or 5:1 (25,000 cells). As a full lysis control, 1 μM of staurosporine was added to both WI-38 and etoposide-treated senescent WI-38 cells. Electrical impedance was measured every 30 min over 24 hours and normalized to the starting value (normalized cell index). For the data analysis, the time-points from 30 min (where the signal is stable) to 6 hours was used. The % cytolysis was calculated using (Eq. 1). Normalized WI-38 and etoposide-treated senescent WI-38 conditions were compared to their corresponding conditions with effector iNKT cells at each time point.

$$\% Cytolysis = \frac{( Cell {Index}_{no effector} - Cell {Index}_{effector} )}{Cell {Index}_{no effector}} \times 100 \qquad Eq . 1$$

QUANTIFICATION AND STATISTICAL ANALYSIS For each experiment, the number of biological replicates (n) is indicated in the figure legend. Statistical comparisons were performed between groups of mice, and/or samples withn≥3 biological replicates or mice, using two-tailed T-tests for two groups, or one-way ANOVA, or two-way ANOVA for three or more groups, with corrections for multiple testing. P<0.05 was considered significant.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

What is claimed is:

1. An agent comprising an iNKT cell activator, iNKT cell, or iNKT cell precursor, for use in a method of reducing the number of senescent cells in a selected target tissue, organ, or compartment of a subject.

2. The agent of claim 1, wherein
the target tissue, organ, or compartment of the subject is selected from: adipose tissue; the lung; the liver; the kidney; the spleen; the brain; cardiovascular tissue; the pancreas; skeletal muscle; and the dermis.

3. An agent comprising an iNKT cell activator, iNKT cell, or iNKT cell precursor, for use in a method of inhibiting a pathological process associated with senescent cells in a selected target tissue, organ, or compartment of a subject.

4. The agent of claim 3, wherein
the target tissue, organ, or compartment of the subject is selected from: adipose tissue; the lung; the liver; the kidney; the spleen; the brain; cardiovascular tissue; the pancreas; skeletal muscle; and the dermis.

5. The agent of claim 3, wherein
the pathological process is inflammation.

6. The agent of claim 5, wherein
the pathological process is inflammation associated with senescent cells exhibiting SASP.

7. The agent of claim 3, wherein
the pathological process is fibrosis.

8. An agent comprising an iNKT cell activator, iNKT cell, or iNKT cell precursor, for use in a method of treating a senescence-associated condition in a subject.

9. The agent of claim 8, wherein
the senescence-associated condition is lung fibrosis.

10. The agent of claim 8, wherein
the senescence-associated condition is diabetes.

11. The agent of claim 8, wherein
the senescence-associated condition is an inflammatory or autoimmune condition.

12. The agent of claim 8, wherein
the senescence-associated condition is an age-related condition.

13. The agent of claim 8, wherein
the senescence-associated condition is a neurodegenerative condition.

14. The agent of claim 8, wherein
the treatment is a preventative treatment.

15. The agent of any of claims 1-14, wherein
the agent comprises an activator of iNKT cells.

16. The agent of claim 15, wherein
the agent comprises a glycolipid.

17. The agent of claim 15, wherein
the glycolipid is alpha-galactosylceramide.

18. The agent of claim 15, wherein
the glycolipid comprises a variant of alpha-galactosylceramide.

19. The agent of claim 15, wherein
the glycolipid is complexed with a CD1d protein or oligomer.

20. The agent of claim 19, wherein
the glycolipid is complexed with a CD1d dimer or tetramer.

21. The agent of any of claims 1-14, wherein
the agent is a CD1d-expressing cell functionalized with or loaded with glycolipid iNKT activators.

22. The agent of claim 21, wherein
the CD1d-expressing cell is a dendritic cell.

23. The agent of any of claims 1-14, wherein
the agent comprises an antibody which selectively binds to and activates the TCR of iNKT cells.

24. The agent of any of claims 1-14, wherein
the agent comprises an iNKT cell or iNKT precursor.

25. A method of treating a senescence associated-condition in a subject in need of treatment therefore by the administration to the subject of a therapeutically effective amount of an agent comprising an iNKT activator, iNKT cell, or iNKT cell precursor, wherein the agent facilitates the removal of senescent cells.

26. The method of claim 25, wherein
the iNKT agent is an iNKT cell activator.

27. The method of claim 25, wherein
the iNKT activator is a glycolipid.

28. The method of claim 27, wherein
the glycolipid is alpha-galactosylceramide.

29. The method of claim 27, wherein
the glycolipid is an alpha-galactosylceramide variant.

30. The method of claim 27, wherein the
the glycolipid is complexed with a CD1d protein or oligomer.

31. The method of claim 30, wherein the
the glycolipid is complexed with a CD1d dimer or tetramer.

32. The method of claim 26, wherein
the iNKT cell activator comprises a CD1d-expressing cell functionalized or loaded with a glycolipid activator of iNKT cells.

33. The method of claim 32, wherein
the CD1d-expressing cell is a dendritic cell.

34. The method of claim 26, wherein the
the iNKT cell activator comprises an antibody which selectively binds to the TCR of iNKT cells.

35. The method of claim 25, wherein the
the agent comprises an iNKT cell or iNKT cell precursor.

36. The method of claim 25, wherein
the senescence-associated condition is lung fibrosis.

37. The method of claim 25, wherein
the senescence-associated condition is diabetes.

38. The agent of claim 25, wherein
the senescence-associated condition is an inflammatory or autoimmune condition.

39. The method of claim 25, wherein
the senescence-associated condition is an age-related condition.

40. The method of claim 25, wherein
the senescence-associated condition is a neurodegenerative condition.

41. The method of claim 25, wherein
the treatment is a preventative treatment.
